(19) United States
(12) Patent Application Publication
(10) Pub. No.: US 2026/0199437 A1
(43) Pub. Date: Jul. 16, 2026

(54) SEMAGLUTIDE FORMULATIONS

(71) Applicant: PROCOMPOUNDING, LLC, Johnson City, TN (US)

(72) Inventors: Jessica Laraine Patrone, Jonesborough, TN (US); Laura Beth Jenks, Abingdon, VA (US)

(21) Appl. No.: 19/442,666

(22) Filed: Jan. 7, 2026

Related U.S. Application Data
(63) Continuation of application No. PCT/US2024/037077, filed on Jul. 8, 2024.
(60) Provisional application No. 63/512,507, filed on Jul. 7, 2023.

(51) Int. Cl. A61K 38/26; A61K 9/00; A61K 31/485; A61K 31/714; A61K 47/12; A61K 47/18; A61K 47/32; A61K 47/38

(52) U.S. Cl. CPC A61K 38/26; A61K 9/0031; A61K 9/006; A61K 31/485; A61K 31/714; A61K 47/12; A61K 47/183; A61K 47/32; A61K 47/38

(57) ABSTRACT

Disclosed herein are semaglutide formulations that comprise lower quantities of semaglutide compared to commercially available products; as well as their methods of making and methods of using.

### CROSS-REFERENCE TO RELATED APPLICATIONS

This application claims the benefit of priority of U.S. provisional application No. 63/512,507, filed Jul. 7, 2023, the disclosure of which is hereby incorporated by reference in its entirety.

### TECHNICAL FIELD

The present application relates to compounded therapies. In particular, the present application relates to sublingual, buccal or rectal formulations including semaglutide and methods for compounding the same.

### BACKGROUND

The development of drug delivery routes remains an important element in the progress of the pharmaceutical sciences. Once an active compound has been identified, the design of delivery mechanisms must overcome challenges of transporting the medicament to the required site of action in the body whilst addressing issues including shelf life, bioavailability, toxicity, and patient compliance. All of these challenges must be overcome to achieve the desired therapeutic effect. Amongst the drug delivery options, oral administration is by far the most common route, with other options including injection, inhalation, topical or transmucosal administration.

The oral delivery route faces perhaps the most challenging route for a pharmaceutical to reach the final site of action: the composition is prone to loss from the mouth or stomach (e.g. by spitting or vomiting); the composition must survive the acidic and enzymatically-active environment of the stomach; if not absorbed in the stomach, the medicament must survive the action of bile salts and further intestinal and bacterial enzymatic action within the intestinal tract, be able to cross from the lumen of the gut to the intestinal wall for absorption, and then survive the degradation processes of the liver following transport by the hepatic portal system, often resulting in poor availability due to the first pass effect. In addition, some patients can't take (or don't like taking) tablets, a common form for oral dosing. Despite these challenges, the oral route of drug administration remains the most common.

### Semaglutide

Rybelsus is the only commercially available orally administrated composition comprising semaglutide, as Ozempic and Wegovy are injectable formulations comprising semaglutide. Rybelsus occurs in only. Rybelsus suffers from the aforementioned challenges as an orally administrated drug, with low bio-availabilities that are estimated to be about 0.8-1.0%.

To offset the low bioavailability of Rybelsus's 3 mg, 7 mg, or 14 mg semaglutide quantities, Novo Nordisk is seeking FDA approval for oral semaglutide for weight loss at 25-50 mg. In a phase 3 trial called PIONEER PLUS, Novo's oral semaglutide at 25-mg and 50-mg doses outperformed the approved 14-mg dose.

Due to low bio-availabilities, there is a need in the art for alternative formulations comprising semaglutide that avoid the drawbacks associated with orally administrated drugs.

### SUMMARY

The disclosure is directed to a sublingual, buccal or rectal formulation comprising 0.25 mg-5.0 mg semaglutide and one or more inactive components, as well as methods of making and using the sublingual, buccal or rectal formulation.

In a variation, the sublingual, buccal or rectal formulation comprises semaglutide and at least one of naltrexone and vitamin B₁₂.

In a variation, the one or more inactive components of the sublingual, buccal or rectal formulation comprises at least one of an anhydrous base, a suspension vehicle, a sweetener, an anti-bitter agent, a mucoadhesive agent, a flavoring agent or a penetration enhancer.

In a variation, the sublingual, buccal or rectal formulation comprises semaglutide and a penetration enhancer.

In a variation, the formulation is a sublingual tablet or sublingual suspension.

In a variation, the formulation is a buccal tablet, buccal wafer, buccal troche or buccal lozenge.

In a variation, the formulation is a suppository.

The disclosure is also directed to a method for treating diabetes and/or obesity in a subject in need of such treatment, said method comprising administering to said subject a therapeutically effective amount of the sublingual, buccal or rectal formulation described herein.

The disclosure is also directed to a method for managing weight in a subject in need of such treatment, said method comprising administering to said subject a therapeutically effective amount of the sublingual, buccal or rectal formulation described herein.

The disclosure is also directed to a method for controlling glucose in a subject in need of such treatment, said method comprising administering to said subject a therapeutically effective amount of the sublingual, buccal or rectal formulation described herein.

The disclosure is also directed to a method for reducing cardiovascular events in an adult with diabetes in a subject in need of such treatment, said method comprising administering to said subject a therapeutically effective amount of the sublingual, buccal or rectal formulation described herein.

In a variation, the formulation for any method of use described herein is administered sublingually.

In a variation, the formulation for any method of use described herein is administered buccally.

In a variation, the formulation for any method of use described herein is administered rectally.

In a variation, the formulation for any method of use described herein is administered at least once a day. In a variation, the formulation for any method of use described herein is administered at least twice a day.

In a variation, the formulation for any method of use described herein is administered at least once a week. In a variation, the formulation for any method of use described herein is administered at least twice a week.

### BRIEF DESCRIPTION OF THE DRAWINGS

FIG. 1A shows the ultra-high performance liquid chromatography-quadrupole time-of-flight mass spectra (UHPLC/TOF-MS) of the 100 ng/mL semaglutide standard. FIG. 1B shows the UHPLC/TOF-MS of the semaglutide study specimen.

FIG. 2 shows a comparison of the kinetic profile of the bioavailability of semaglutide over time.

FIG. 3 shows a comparison of steady state bioavailability of the injectable formulation and of the troche formulation after several blood drawings.

FIG. 4 shows the UHPLC/TOF-MS of a semaglutide troche formulation.

### DETAILED DESCRIPTION

The formulations described herein may be manufactured with the appropriate grade of ingredients. For instance, it may be desirable to prepare the formulations described herein with USP, NF or EP grade ingredients.

As used herein “USP grade” is taken to mean ingredients manufactured under current Good Manufacturing Practices (cGMP) and which meet the requirements of the US Pharmacopeia (USP).

As used herein “NF grade” is taken to mean ingredients manufactured under current Good Manufacturing Practices (cGMP) and meeting the requirements of the National Formulary (NF).

As used herein “EP grade” is taken to mean ingredients that meet the specifications developed by the European pharmacopoeia (EP).

Semaglutide is currently FDA approved and can be administered either by injection or orally as RYBELSUS®.

RYBELSUS® contains 3 mg, 7 mg, or 14 mg semaglutide, and inactive ingredients: magnesium stearate, microcrystalline cellulose (MCC), povidone and salcaprozate sodium (SNAC).

The disclosure provides semaglutide for administration by sublingual, buccal or rectal modes of administration. The amount of semaglutide for administration can be substantially less than the approved quantity in RYBELSUS® for sublingual, buccal or rectal formulations.

### A. Semaglutide

Semagluide refers to the compound of Formula (I):

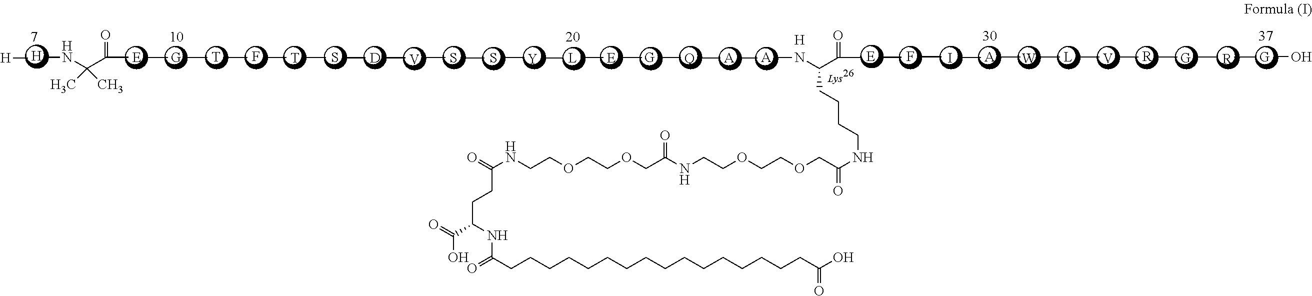

Semaglutide is a GLP-1 receptor agonist. The peptide backbone is produced by yeast fermentation. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by the enzyme dipeptidyl-peptidase 4 (DPP-4). A minor modification was made in position 34 to ensure the attachment of only one fatty di-acid. The molecular formula is C₁₈₇H₂₉₁N₄₅O₅₉ and the molecular weight is 4113.58 g/mol.

The disclosure provides for semaglutide formulations that comprise semaglutide in smaller quantities than the approved for oral administration. In some variations, the quantity of semaglutide in a dose is from 0.25 mg-5.0 mg. In some variations, the quantity of semaglutide is at least 0.25 mg. In some variations, the quantity of semaglutide is at least 0.50 mg. In some variations, the quantity of semaglutide is at least 1.0 mg. In some variations, the quantity of semaglutide is at least 1.5 mg. In some variations, the quantity of semaglutide is at least 2.0 mg. In some variations, the quantity of semaglutide is at least 2.5 mg. In some variations, the quantity of semaglutide is at least 3.0 mg. In some variations, the quantity of semaglutide is at least 3.5 mg. In some variations, the quantity of semaglutide is at least 4.0 mg. In some variations, the quantity of semaglutide is at least 4.5 mg. In some variations, the quantity of semaglutide is at least 5.0 mg.

In some variations, the amount of semaglutide in a dose is less than or equal to 5.0 mg. In some variations, the amount of semaglutide in a dose is less than or equal to 4.5 mg. In some variations, the amount of semaglutide in a dose is less than or equal to 4.0 mg. In some variations, the amount of semaglutide in a dose is less than or equal to 3.5 mg. In some variations, the amount of semaglutide in a dose is less than or equal to 3.0 mg. In some variations, the amount of semaglutide in a dose is less than or equal to 2.5 mg. In some variations, the amount of semaglutide in a dose is less than or equal to 2.0 mg. In some variations, the amount of semaglutide in a dose is less than or equal to 1.5 mg. In some variations, the amount of semaglutide in a dose is less than or equal to 1.0 mg. In some variations, the amount of semaglutide in a dose is less than or equal to 0.5 mg. In some variations, the amount of semaglutide in a dose is less than or equal to 0.25 mg.

It would be understood that any lower quantity of semaglutide can be combined with any upper quantity of semaglutide in any combination described herein.

It would be understood that in order to achieve the intended therapeutic results, multiple dosing per day (or per week) of the formulations described herein may be necessary, for instance, due to concerns about half-life of accumulative effects of the active ingredients. For instance, any of the formulations described herein may be dosed once daily or more than once daily, such as twice daily or three times daily. For instance, any of the formulations described herein may be dosed once weekly or more than once weekly, such as twice weekly or three times weekly.

### B. Modes of Administration

### 1. Sublingual Administration

Sublingual literally means under the tongue and refers to a method of administering substances via the mouth in such a way that the substances are rapidly absorbed via the blood vessels under the tongue rather than via the digestive tract. Sublingual medications don't need to be chewed or swallowed to be absorbed by the body. Instead, they dissolve under the tongue.

Sublingual products come in a variety of forms, including tablets, oral disintegrating tablets, wafers, suspensions, solutions, films, sprays, lozenges, and troches.

In some variations, the sublingual formulations described herein can be in the form of a tablet. Sublingual tablets are placed beneath the tongue, where the drug substance is absorbed directly through the oral mucosa.

Methods of preparing sublingual tablets are known to those skilled in the art and include freeze-drying, granulation, compression and/or baking.

Most sublingual tablets include at least one active ingredient and a number of excipients. These excipients may include fillers (diluents), binders, disintegrating agents, lubricants, and glidants. Approved FD&C and D&C dyes or lakes, flavors, and sweetening agents also may be included.

Fillers or diluents are added when the quantity of active ingredient(s) is too small, or the properties of the active ingredient(s) do not allow satisfactory compaction in the absence of other ingredients. Binders impart adhesiveness to the powder blend and promote tablet formation and maintenance of drug substance uniformity in the tableting mixture. Disintegrating agents facilitate reduction of the tablet into small particles upon contact with water or biological fluids. Lubricants reduce friction during the compaction and ejection cycles. Glidants improve powder fluidity, powder handling properties, and tablet weight control. Colorants are often added to tablet formulations for aesthetic value or for product identification.

Tablets are prepared from formulations that have been processed by one of three general methods: wet granulation, dry granulation (roll compaction or slugging), or direct compression.

Wet granulation involves the mixing of dry powders with a granulating liquid to form a moist granular mass that is dried and sized prior to compression. It is particularly useful in achieving uniform blends of low-dose drug substances and facilitating the wetting and dissolution of poorly soluble, hydrophobic drug substances.

Dry granulation involves passing powders between rollers at elevated pressure (roll compaction). Alternatively, dry granulation also can be carried out by the compaction of powders at high pressures on tablet presses, a process also known as slugging. In either case, the compacts are sized before compression. Dry granulation improves the flow and handling properties of the powder formulation without involving moisture in the processing.

Direct compression involves dry blending of the active ingredient(s) and excipients followed by compression. The simplest manufacturing technique, direct compression, is acceptable when the drug substance and excipients possess acceptable flow and compression properties without prior process steps.

Tablets may be coated to protect the ingredients from air, moisture, or light; to mask unpleasant tastes and odors; to improve tablet appearance; and to reduce dustiness. In addition, coating may be used to protect the drug substance from acidic pH values associated with gastric fluids or to control the rate of drug release in the gastrointestinal tract. A common coating is a thin film coating composed of a polymer that is derived from cellulose. Sugar coating is an alternative, less common approach. Sugar-coated tablets have considerably thicker coatings that are primarily sucrose with a number of inorganic diluents. A variety of film-coating polymers are available and enable the development of specialized release profiles. These formulations are used to protect acid-labile drug substances from the acidic stomach environment as well as to prolong the release of the drug substance to reduce dosing frequency

Sublingual tablets described herein may include the following inactive ingredients, without limitation. Such inactive ingredients may include lactose, glyceryl behenate, glyceryl exosadioate, sodium starch glycolate, calcium stearate, acacia, mannitol, starch, microcrystalline cellulose, magnesium stearate and saccharin.

In some variations, the sublingual tablets include lactose, starch, microcrystalline cellulose or magnesium stearate.

In some variations, the sublingual formulation can be in the form of an orally disintegrating tablet.

Methods of preparing orally disintegrating sublingual tablets are known to those skilled in the art and include freeze-drying, granulating compression and/or baking.

In some variations, the sublingual formulation can be in the form of a wafer.

In some variations, the sublingual formulation can be in the form of a suspension.

Methods of preparing sublingual suspensions are known to those skilled in the art. For instance, suspensions are prepared by adding suspending agents or other excipients and purified water or oil (e.g., medium chain triglycerides (MCT)) to solid active ingredient(s) and mixing to achieve uniformity. In the preparation of a suspension, the characteristics of both the dispersed phase and the dispersion medium should be considered. During development, the appropriate particle size distribution for the suspended material should be defined to achieve the desired effectiveness and to minimize the likelihood of particle size changes during storage.

In some instances, the dispersed phase has an affinity for the vehicle and is readily wetted upon its addition. For some materials, the displacement of air from the solid surface is difficult, and the solid particles may clump together or float on top of the vehicle. In the latter case, a wetting agent may be used for certain types of suspensions to facilitate displacement of air from the powder surface. Surfactants, alcohol, glycerin, and other hydrophilic liquids can be used as wetting agents when an aqueous vehicle will be used as the dispersion phase. These agents function by displacing the air in the crevices of the particles and dispersing the particles. In the large-scale preparation of suspensions, wetting of the dispersed phase may be aided by the use of high-energy mixing equipment such as colloid mills or other rotor-stator mixing devices.

After the powder has been wetted, the dispersion medium (containing the soluble formulation components such as colorants, flavorings, and preservatives) is added in portions to the powder, and the mixture is thoroughly blended before subsequent additions of the vehicle. A portion of the vehicle is used to wash the mixing equipment free of suspended material, and this portion is used to bring the suspension to final volume and ensure that the suspension contains the desired concentration of solid matter. The final product may be passed through a colloid mill or other blender or mixing device to ensure uniformity.

In some variations, the sublingual formulation can be in the form of a solution.

In some variations, the sublingual formulation can be in the form of a film. Sublingual films are placed beneath the tongue, where the drug substance is absorbed directly through the oral mucosa. Sublingual films can be formulated with edible polymers such as pullulan or with water-soluble polymers such as modified cellulose, edible gums, and copolymers. The dissolution rate of the film is controlled to facilitate incorporation of the medication into saliva or for absorption by the proximal mucosa. These films must be substantial enough to maintain their integrity during manufacture and packaging, and permit handling by the patient. Because of the rapid dissolution, taste and mouth feel are important considerations.

Sublingual films described herein may include the following inactive ingredients, without limitation. Such inactive ingredients are polyethylene glycol, Hypromellose and maltitol.

In some variations, the sublingual films include polyethylene glycol or Hypromellose.

In some variations, the sublingual formulation can be in the form of a spray. Examples of a spray may include sprayable solutions or sprayable suspensions.

In some variations, the sublingual formulation can be in the form of a lozenge. Lozenges are solid dosage forms that are designed to dissolve or disintegrate slowly in the mouth. They contain one or more active ingredient(s) that are slowly liberated from the typically flavored and sweetened base. They are frequently intended to provide local action in the oral cavity or the throat but also include those intended for systemic absorption after dissolution.

Lozenges prepared by compression or by stamping or cutting from a uniform bed of paste are sometimes known as troches (a term not used in official article titles). Compressed or stamped lozenges are often produced in a circular shape. Lozenges can be made using sugars such as sucrose and dextrose or can provide the benefits of a sugar-free formulation that is usually based on sorbitol or mannitol. Polyethylene glycols and hypromellose are sometimes included to slow the rate of dissolution.

Excipients used in molded lozenge manufacture may include gelatin, fused sucrose, sorbitol, or another carbohydrate base. Molded lozenges can be prepared by mixing the ingredients with water and heating to reduce the water content. The viscous solution is then poured into molds (e.g., corn starch molds). The lozenges are quickly cooled in the molds to trap the base in the glassy state. Once formed, the lozenges are removed from the molds and packaged. Care is taken to avoid excessive moisture during storage to prevent crystallization of the sugar base.

Compressed lozenges are made using excipients that may include a filler, binder, sweetening agent, flavoring agent, and lubricant. Sugars such as sucrose, sorbitol, and mannitol are often included because they can act as a filler and binder as well as serve as sweetening agents. Approved FD&C and D&C dyes or lakes (dyes adsorbed onto insoluble aluminum hydroxide) may also be included.

The manufacturing of compressed lozenges is essentially the same as that for conventional tableting, with the exception that a tablet press capable of making larger tablets and exerting greater force to produce harder tablets may be required. The paste used to produce lozenges manufactured by stamping or cutting contains a moistening agent, sucrose, and flavoring and sweetening agents. The homogenous paste is spread as a bed of uniform thickness, and the lozenges are cut or stamped from the bed and are allowed to dry. Some lozenges are prepared by forcing dampened powders under low pressure into mold cavities and then ejecting them onto suitable trays for drying at moderate temperatures.

Sublingual lozenges described herein may include the following inactive ingredients, without limitation. Such inactive ingredients are magnesium stearate, microcrystalline cellulose, dextrose and ascorbic acid.

In some variations, the sublingual lozenge include magnesium stearate.

In some variations, the sublingual formulation can be in the form of a troche. Sublingual troches described herein may include the following inactive ingredients, without limitation. Such inactive ingredients are polyethylene glycol, glycerinated gelatin (glycerin and gelatin), acacia, polacrilin, calcium polycarbophil, mannitol and xanthum gum.

In some variations, the sublingual troche include polyethylene glycol or glycerinated gelatin (glycerin and gelatin).

### 2. Buccal Administration

Sublingual administration involves placing a drug under the tongue to dissolve and absorb into the blood through the mucosa tissue, whereas buccal administration involves placing a drug between the gums and cheek, where it also dissolves and is absorbed into your blood. Buccal products come in a variety of forms, including tablets, oral disintegrating tablets, wafers, suspensions, solutions, films, sprays, lozenges, and troches.

Unless otherwise specified, the buccal formulations described herein are substantially similar to the sublingual formulations described herein. In other words, and unless otherwise specified, “sublingual” and “buccal” may be used interchangeably. The variations described herein for sublingual formulations are equally applicable to buccal formulations.

### 3. Rectal Administration

Rectal administration uses the rectum as a route of administration for medication and other fluids, which are absorbed by the rectum's blood vessels and flow into the body's circulatory system, which distributes the drug to the body's organs and bodily systems.

In some variations, the rectal formulations described herein can be in the form of a suppository.

Suppository bases may include cocoa butter, glycerinated gelatin, hydrogenated vegetable oils, mixtures of polyethylene glycols of various molecular weights, and fatty acid esters of polyethylene glycol. The suppository base can have a notable influence on the release of the drug substance(s). Although cocoa butter melts quickly at body temperature, it is immiscible with body fluids, and this inhibits the diffusion of fat-soluble drug substances to the affected sites. Polyethylene glycol is a suitable base for some antiseptics. In cases when systemic action is desired, incorporating the ionized rather than the nonionized form of the drug substance may help maximize bioavailability. Although nonionized drug substances partition more readily out of water-miscible bases such as glycerinated gelatin and polyethylene glycol, the bases themselves tend to dissolve very slowly, which slows drug substance release. Cocoa butter and its substitutes (e.g., Hard Fat) perform better than other bases for allaying irritation in preparations intended for treating internal hemorrhoids. Suppositories for adults are tapered at one or both ends and usually weigh about 2 g each.

Methods of preparing suppositories are known to those skilled in the art.

Cocoa butter suppositories have cocoa butter as the base and can be made by incorporating the finely divided drug substance into the solid oil at room temperature and suitably shaping the resulting mass, or by working with the oil in the melted state and allowing the resulting suspension to cool in molds. A suitable quantity of hardening agents may be added to counteract the tendency of some drug substances (such as chloral hydrate and phenol) to soften the base. The finished suppository melts at body temperature.

A variety of vegetable oils, such as coconut or palm kernel, modified by esterification, hydrogenation, or fractionation, are used as cocoa butter substitutes to obtain products that display varying compositions and melting temperatures (e.g., Hydrogenated Vegetable Oil and Hard Fat). These products can be designed to reduce rancidity while incorporating desired characteristics such as narrow intervals between melting and solidification temperatures and melting ranges to accommodate formulation and climatic conditions.

Active ingredient(s) can be incorporated into glycerinated gelatin bases by addition of the prescribed quantities to a vehicle consisting of about 70 parts of glycerin, 20 parts of gelatin, and 10 parts of water.

Several combinations of polyethylene glycols that have melting temperatures that are above body temperature are used as suppository bases. Because release from these bases depends on dissolution rather than on melting, there are significantly fewer problems in preparation and storage than is the case for melting-type vehicles. However, high concentrations of higher molecular weight polyethylene glycols may lengthen dissolution time, resulting in problems with retention.

Several nonionic surface-active agents closely related chemically to the polyethylene glycols can be used as suppository vehicles. Examples include polyoxyethylene sorbitan fatty acid esters and the polyoxyethylene stearates. These surfactants are used alone or in combination with other suppository vehicles to yield a wide range of melting temperatures and consistencies. A notable advantage of such vehicles is their water dispersibility. However, care must be taken with the use of surfactants because they may either increase the rate of drug substance absorption or interact with the drug substance to reduce therapeutic activity.

Compounding suppositories using a suppository base typically involves melting the suppository base and dissolution or dispersion of the drug substance in the molten base. When compounding suppositories, the compounding professional prepares an excess amount of total formulation to allow the prescribed quantity to be accurately dispensed. In compounding suppositories, avoid caustic or irritating ingredients, carefully select a base that will allow the drug substance to intended effect, and in order to minimize abrasion of the rectal membranes, reduce solid ingredients to the smallest sonable particle size.

Suppositories described herein may include the following inactive ingredients, without limitation. Such inactive ingredients are cocoa butter, vegetable oil, glycerinated gelatin (glycerin and gelatin), hydrogenated vegetable oil, polyethylene glycol with high molecular weight, polyethylene glycol with low molecular weight, fatty acid esters of polyethylene glycol, glycerin and coconut oil.

In some variations, the suppositories described herein include polyethylene glycol with high molecular weight, fatty acid esters of polyethylene glycol or glycerinated gelatin (glycerin and gelatin).

### C. Additional Active Components

### 1. Naltrexone

Naltrexone is a medication approved by the Food and Drug Administration (FDA) to treat both alcohol use disorder (AUD) and opioid use disorder (OUD), as well as being approved for weight loss. The disclosure provides for naltrexone as an additional active component in the sublingual, buccal and rectal formulations disclosed herein.

In some variations, the quantity of naltrexone in a dose is from 0.0005 mg-50.0 mg. In some variations, the quantity of naltrexone is at least 0.0005 mg. In some variations, the quantity of naltrexone is at least 0.005 mg. In some variations, the quantity of naltrexone is at least 0.05 mg. In some variations, the quantity of naltrexone is at least 0.5 mg. In some variations, the quantity of naltrexone is at least 1.0 mg. In In some variations, the quantity of naltrexone is at least 5.0 mg. In some variations, the quantity of naltrexone is at least 10.0 mg. In some variations, the quantity of naltrexone is at least 15.0 mg. In some variations, the quantity of naltrexone is at least 20.0 mg. In some variations, the quantity of naltrexone is at least 25.0 mg. In some variations, the quantity of naltrexone is at least 30.0 mg. In some variations, the quantity of naltrexone is at least 35.0 mg. In some variations, the quantity of naltrexone is at least 40.0 mg. In some variations, the quantity of naltrexone is at least 45.0 mg. In some variations, the quantity of naltrexone is at least 50.0 mg.

In some variations, the amount of naltrexone in a dose is less than or equal to 50.0 mg. In some variations, the amount of naltrexone in a dose is less than or equal to 45.0 mg. In some variations, the amount of naltrexone in a dose is less than or equal to 40.0 mg. In some variations, the amount of naltrexone in a dose is less than or equal to 35.0 mg. In some variations, the amount of naltrexone in a dose is less than or equal to 30.0 mg. In some variations, the amount of naltrexone in a dose is less than or equal to 25.0 mg. In some variations, the amount of naltrexone in a dose is less than or equal to 20.0 mg. In some variations, the amount of naltrexone in a dose is less than or equal to 15.0 mg. In some variations, the amount of naltrexone in a dose is less than or equal to 10.0 mg. In some variations, the amount of naltrexone in a dose is less than or equal to 5.0 mg. In some variations, the amount of naltrexone in a dose is less than or equal to 1.0 mg. In some variations, the amount of naltrexone in a dose is less than or equal to 0.5 mg. In some variations, the amount of naltrexone in a dose is less than or equal to 0.05 mg. In some variations, the amount of naltrexone in a dose is less than or equal to 0.005 mg. In some variations, the amount of naltrexone in a dose is less than or equal to 0.0005 mg.

It would be understood that any lower quantity of naltrexone can be combined with any upper quantity of naltrexone in any combination described herein.

### Dosage for Autoimmune Disorder

The disclosure provides for naltrexone as an additional active component in the sublingual and buccal formulations disclosed herein for treating autoimmune disorders. In treating autoimmune disorders, the quantity of naltrexone is as follows.

In some variations, the quantity of naltrexone in a dose is from 1.5 mg-4.0 mg. In some variations, the quantity of naltrexone is at least 1.5 mg. In some variations, the quantity of naltrexone is at least 1.75 mg. In some variations, the quantity of naltrexone is at least 2.0 mg. In some variations, the quantity of naltrexone is at least 2.25 mg. In some variations, the quantity of naltrexone is at least 2.5 mg. In some variations, the quantity of naltrexone is at least 2.75 mg. In some variations, the quantity of naltrexone is at least 3.0 mg. In some variations, the quantity of naltrexone is at least 3.25 mg. In some variations, the quantity of naltrexone is at least 3.5 mg. In some variations, the quantity of naltrexone is at least 3.75 mg. In some variations, the quantity of naltrexone is at least 4.0 mg.

In some variations, the amount of naltrexone in a dose is less than or equal to 4.0 mg. In some variations, the amount of naltrexone in a dose is less than or equal to 3.75 mg. In some variations, the amount of naltrexone in a dose is less than or equal to 3.50 mg. In some variations, the amount of naltrexone in a dose is less than or equal to 3.25 mg. In some variations, the amount of naltrexone in a dose is less than or equal to 3.00 mg. In some variations, the amount of naltrexone in a dose is less than or equal to 2.75 mg. In some variations, the amount of naltrexone in a dose is less than or equal to 2.50 mg. In some variations, the amount of naltrexone in a dose is less than or equal to 2.25 mg. In some variations, the amount of naltrexone in a dose is less than or equal to 2.00 mg. In some variations, the amount of naltrexone in a dose is less than or equal to 1.75 mg. In some variations, the amount of naltrexone in a dose is less than or equal to 1.50 mg.

It would be understood that any lower quantity of naltrexone can be combined with any upper quantity of naltrexone in any combination described herein.

### Dosage for Reducing Food Cravings or Promoting Weight Loss

The disclosure provides for naltrexone as an additional active component in the sublingual and buccal formulations disclosed herein for reducing food cravings or for promoting weight loss. In treating food cravings, or promoting weight loss, the quantity of naltrexone is as follows.

In some variations, the quantity of naltrexone in a dose is from 8.0 mg-32.0 mg. In some variations, the quantity of naltrexone is at least 8.0 mg. In some variations, the quantity of naltrexone is at least 9.0 mg. In some variations, the quantity of naltrexone is at least 10.0 mg. In some variations, the quantity of naltrexone is at least 11.0 mg. In some variations, the quantity of naltrexone is at least 12.0 mg. In some variations, the quantity of naltrexone is at least 13.0 mg. In some variations, the quantity of naltrexone is at least 14.0 mg. In some variations, the quantity of naltrexone is at least 15.0 mg. In some variations, the quantity of naltrexone is at least 16.0 mg. In some variations, the quantity of naltrexone is at least 17.0 mg. In some variations, the quantity of naltrexone is at least 18.0 mg. In some variations, the quantity of naltrexone is at least 19.0 mg. In some variations, the quantity of naltrexone is at least 20.0 mg. In some variations, the quantity of naltrexone is at least 21.0 mg. In some variations, the quantity of naltrexone is at least 22.0 mg. In some variations, the quantity of naltrexone is at least 23.0 mg. In some variations, the quantity of naltrexone is at least 24.0 mg. In some variations, the quantity of naltrexone is at least 25.0 mg. In some variations, the quantity of naltrexone is at least 26.0 mg. In some variations, the quantity of naltrexone is at least 27.0 mg. In some variations, the quantity of naltrexone is at least 28.0 mg. In some variations, the quantity of naltrexone is at least 29.0 mg. In some variations, the quantity of naltrexone is at least 30.0 mg. In some variations, the quantity of naltrexone is at least 31.0 mg. In some variations, the quantity of naltrexone is at least 32.0 mg.

In some variations, the amount of naltrexone in a dose is less than or equal to 32.0 mg. In some variations, the amount of naltrexone in a dose is less than or equal to 31.0 mg. In some variations, the amount of naltrexone in a dose is less than or equal to 30.0 mg. In some variations, the amount of naltrexone in a dose is less than or equal to 29.0 mg. In some variations, the amount of naltrexone in a dose is less than or equal to 28.0 mg. In some variations, the amount of naltrexone in a dose is less than or equal to 27.0 mg. In some variations, the amount of naltrexone in a dose is less than or equal to 26.0 mg. In some variations, the amount of naltrexone in a dose is less than or equal to 25.0 mg. In some variations, the amount of naltrexone in a dose is less than or equal to 24.0 mg. In some variations, the amount of naltrexone in a dose is less than or equal to 23.0 mg. In some variations, the amount of naltrexone in a dose is less than or equal to 22.0 mg. In some variations, the amount of naltrexone in a dose is less than or equal to 21.0 mg. In some variations, the amount of naltrexone in a dose is less than or equal to 20.0 mg. In some variations, the amount of naltrexone in a dose is less than or equal to 19.0 mg. In some variations, the amount of naltrexone in a dose is less than or equal to 18.0 mg. In some variations, the amount of naltrexone in a dose is less than or equal to 17.0 mg. In some variations, the amount of naltrexone in a dose is less than or equal to 16.0 mg. In some variations, the amount of naltrexone in a dose is less than or equal to 15.0 mg. In some variations, the amount of naltrexone in a dose is less than or equal to 14.0 mg. In some variations, the amount of naltrexone in a dose is less than or equal to 13.0 mg. In some variations, the amount of naltrexone in a dose is less than or equal to 12.0 mg. In some variations, the amount of naltrexone in a dose is less than or equal to 11.0 mg. In some variations, the amount of naltrexone in a dose is less than or equal to 10.0 mg. In some variations, the amount of naltrexone in a dose is less than or equal to 9.0 mg. In some variations, the amount of naltrexone in a dose is less than or equal to 8.0 mg.

It would be understood that any lower quantity of naltrexone can be combined with any upper quantity of naltrexone in any combination described herein.

### Dosage for Treating Alcohol Dependence

The disclosure provides for naltrexone as an additional active component in the sublingual and buccal formulations disclosed herein for treating alcohol dependence. In treating alcohol dependence, the quantity of naltrexone is as follows.

In some variations, the quantity of naltrexone in a dose is about 50.0 mg.

2. Compounds that Reduce Cravings

In some variations, it would be desirable or beneficial to include compounds that reduce cravings in the sublingual, buccal and rectal formulations described herein.

In some variations, compounds that reduce cravings are herbs or vitamins.

Such herbs and vitamins may include, without limitation, 5-htp, b vitamins, magnesium, chromium picolinate. Each of the 5-htp, b vitamins, magnesium picolinate and chromium picolinate may be included in the formulations described herein. For instance, chromium picolinate may be included such that a dosage of 200-1000 mcg daily is realized, such as 600 mcg daily.

Compounds known to control appetite may also be included in the formulations described herein. For instance, GABA (gamma-aminobutyric acid) may be included in the formulations described herein.

Compounds known to increase metabolism may also be included in the formulations described herein. For instance, zinc, green tea, green coffee and/or bitter orange may be included in the formulations described herein.

Compounds known to control sugar metabolism may also be included in the formulations described herein. For instance, zinc, fish oil (omega-3), 1-glutamine, chromium picolinate and/or alpha-lipoic acid may be included in the formulations described herein.

Compounds known to reduce hunger may also be included in the formulations described herein. For instance, GABA, chromium picolinate and/or glucomannan may be included in the formulations described herein.

Compounds known to promote digestion may also be included in the formulations described herein. For instance, lipase may be included in the formulations described herein.

In some variations, compounds that reduce cravings are medications.

Such medications may include, without limitation, naltrexone. For reduction in cravings, the formulations described herein may include 1 mg-50 mg of naltrexone.

Such medications may include, without limitation, antidepressants such as bupropion, fluoxetine and duloxetine. For reduction in cravings, the formulations described herein may include 75 mg-450 mg of bupoprion. For reduction in cravings, the formulations described herein may include 10 mg-40 mg of fluoxetine. For reduction in cravings, the formulations described herein may include 20 mg-120 mg of duloxetine.

Such medications may include, without limitation, emetics such as apomorphine. For reduction in cravings, the formulations described herein may include 1 mg-6 mg of apomorphine.

Such medications may include, without limitation, stimulants such as phentermine, phendimetrazine, amphetamine salts, benzphetamine and/or diethylproprion. For reduction in cravings, the formulations described herein may include 3 mg-37.5 mg of phentermine. For reduction in cravings, the formulations described herein may include 35 mg-105 mg of phendimetrazine. For reduction in cravings, the formulations described herein may include 5 mg-60 mg of amphetamine salts. For reduction in cravings, the formulations described herein may include 25 mg-150 mg of benzphetamine. For reduction in cravings, the formulations described herein may include 25 mg-75 mg of diethylproprion.

Such medications may include, without limitation, a GLP-1 agonist such as liraglutide and/or exenatide. For reduction in cravings, the formulations described herein may include 0.6 mg-3 mg of liraglutide. For reduction in cravings, the formulations described herein may include 5 mcg-20 mcg of exenatide.

Such medications may include, without limitation, topiramate, orlistat and/or setmelanotide. For reduction in cravings, the formulations described herein may include 20 mg -150 mg of topiramate. For reduction in cravings, the formulations described herein may include 75 mg-300 mg of orlistat. For reduction in cravings, the formulations described herein may include 0.5 mg-2 mg of setmelanotide.

Such medications may include, without limitation, a growth hormone-releasing hormone (GHRH) such as sermorelin. For reduction in cravings, the formulations described herein may include 100 mcg-1200 mcg of sermorelin.

### 3. Vitamin B₁₂

The dosage form can include Vitamin B₁₂ or methylated Vitamin B₁₂. Vitamin B₁₂ is not fat soluble. As such, excess clears readily from the body.

The disclosure provides for Vitamin B₁₂ or methylated Vitamin B₁₂ as an additional active component in the sublingual, buccal and rectal formulations disclosed herein. The quantity of Vitamin B₁₂ or methylated Vitamin B₁₂ is as follows. In some variations, the quantity of Vitamin B₁₂ or methylated Vitamin B₁₂ in a dose is from 100.0 mcg-2500.0 mcg. The ranges disclosed below are equally applicable to methylated Vitamin B₁₂.

In some variations, the quantity of Vitamin B₁₂ is at least 100.0 mcg. In some variations, the quantity of Vitamin B₁₂ is at least 200.0 mcg. In some variations, the quantity of Vitamin B₁₂ is at least 300.0 mcg. In some variations, the quantity of Vitamin B₁₂ is at least 400.0 mcg. In some variations, the quantity of Vitamin B₁₂ is at least 500.0 mcg. In some variations, the quantity of Vitamin B₁₂ is at least 600.0 mcg. In some variations, the quantity of Vitamin B₁₂ is at least 700.0 mcg. In some variations, the quantity of Vitamin B₁₂ is at least 800.0 mcg. In some variations, the quantity of Vitamin B₁₂ is at least 900.0 mcg. In some variations, the quantity of Vitamin B₁₂ is at least 1000.0 mcg. In some variations, the quantity of Vitamin B₁₂ is at least 1100.0 mcg. In some variations, the quantity of Vitamin B₁₂ is at least 1200.0 mcg. In some variations, the quantity of Vitamin B₁₂ is at least 1300.0 mcg. In some variations, the quantity of Vitamin B₁₂ is at least 1400.0 mcg. In some variations, the quantity of Vitamin B₁₂ is at least 1500.0 mcg. In some variations, the quantity of Vitamin B₁₂ is at least 1600.0 mcg. In some variations, the quantity of Vitamin B₁₂ is at least 1700.0 mcg. In some variations, the quantity of Vitamin B₁₂ is at least 1800.0 mcg. In some variations, the quantity of Vitamin B₁₂ is at least 1900.0 mcg. In some variations, the quantity of Vitamin B₁₂ is at least 2000.0 mcg. In some variations, the quantity of Vitamin B₁₂ is at least 2100.0 mcg. In some variations, the quantity of Vitamin B₁₂ is at least 2200.0 mcg. In some variations, the quantity of Vitamin B₁₂ is at least 2300.0 mcg. In some variations, the quantity of Vitamin B₁₂ is at least 2400.0 mcg. In some variations, the quantity of Vitamin B₁₂ is at least 2500.0 mcg.

In some variations, the amount of Vitamin B₁₂ in a dose is less than or equal to 2500.0 mcg. In some variations, the amount of Vitamin B₁₂ in a dose is less than or equal to 2400.0 mcg. In some variations, the amount of Vitamin B₁₂ in a dose is less than or equal to 2300.0 mcg. In some variations, the amount of Vitamin B₁₂ in a dose is less than or equal to 2200.0 mcg. In some variations, the amount of Vitamin B₁₂ in a dose is less than or equal to 2100.0 mcg. In some variations, the amount of Vitamin B₁₂ in a dose is less than or equal to 2000.0 mcg. In some variations, the amount of Vitamin B₁₂ in a dose is less than or equal to 1900.0 mcg. In some variations, the amount of Vitamin B₁₂ in a dose is less than or equal to 1800.0 mcg. In some variations, the amount of Vitamin B₁₂ in a dose is less than or equal to 1700.0 mcg. In some variations, the amount of Vitamin B₁₂ in a dose is less than or equal to 1600.0 mcg. In some variations, the amount of Vitamin B₁₂ in a dose is less than or equal to 1500.0 mcg. In some variations, the amount of Vitamin B₁₂ in a dose is less than or equal to 1400.0 mcg. In some variations, the amount of Vitamin B₁₂ in a dose is less than or equal to 1300.0 mcg. In some variations, the amount of Vitamin B₁₂ in a dose is less than or equal to 1200.0 mcg. In some variations, the amount of Vitamin B₁₂ in a dose is less than or equal to 1100.0 mcg. In some variations, the amount of Vitamin B₁₂ in a dose is less than or equal to 1000.0 mcg. In some variations, the amount of Vitamin B₁₂ in a dose is less than or equal to 900.0 mcg. In some variations, the amount of Vitamin B₁₂ in a dose is less than or equal to 800.0 mcg. In some variations, the amount of Vitamin B₁₂ in a dose is less than or equal to 700.0 mcg. In some variations, the amount of Vitamin B₁₂ in a dose is less than or equal to 600.0 mcg. In some variations, the amount of Vitamin B₁₂ in a dose is less than or equal to 500.0 mcg. In some variations, the amount of Vitamin B₁₂ in a dose is less than or equal to 400.0 mcg. In some variations, the amount of Vitamin B₁₂ in a dose is less than or equal to 300.0 mcg. In some variations, the amount of Vitamin B₁₂ in a dose is less than or equal to 200.0 mcg. In some variations, the amount of Vitamin B₁₂ in a dose is less than or equal to 100.0 mcg.

It would be understood that any lower quantity of Vitamin B₁₂ or methylated Vitamin B₁₂ can be combined with any upper quantity of Vitamin B₁₂ or methylated Vitamin B₁₂ in in any combination described herein.

### D. Inactive Components

In some variations, the formulation can include an anhydrous base. Non-limiting examples of anhydrous bases include water, microcrystalline cellulose, carboxymethylcellulose, xanthum gum, acacia, Spray-Dried Powder, NF, Coconut Oil, Refined, Corn Oil, NF, Cottonseed Oil, NF, Light Mineral Oil, NF, Mineral Oil, Heavy, USP, Olive Oil, NF, Polyethylene Glycol 1450, NF, Polyethylene Glycol 1000, NF, Polyethylene Glycol 300, NF, Polyethylene Glycol 3350, USP, Polyethylene Glycol 400, NF, Polyethylene Glycol 8000, NF, Sesame Oil, NF, Syrup, NF, Almond Oil, Sweet, NF, Cod Liver Oil, USP, Croton Oil, Peppermint Oil, NF, Spearmint Oil, Natural, and FCC (all available from Spectrum Chemicals).

In some variations, the anhydrous base is a vegetable oil, Polyethylene Glycol 1450, NF, Polyethylene Glycol 1000, NF, Polyethylene Glycol 300, NF, Polyethylene Glycol 3350, USP, Polyethylene Glycol 400, NF, Polyethylene Glycol 8000, Syrup, NF, water, microcrystalline cellulose, carboxymethylcellulose or xanthum gum.

In some variations, the formulation can include an oral suspension vehicle. Non-limiting examples are silica gel, syrup, cellulose, hypromellulose, ORAL MIX (Flavoured Suspending Vehicle), ORAL MIX, SF (Sugar-Free Flavoured Suspending Vehicle), ORAL SUSPEND (Suspending Vehicle), ORAL SYRUP (Flavored Syrup Vehicle), ORAL SYRUP, SF (Sugar-Free Flavoured Syrup Vehicle), SYRUP, NF (Simple), ORA BLEND® (Flavored Suspending Vehicle), ORA BLEND® SF (Sugar-Free Flavored Oral Suspending Vehicle), ORA PLUS® (Oral Suspending Vehicle), ORA SWEET® (Oral Syrup Vehicle), ORA SWEET® SF (Sugar-Free Oral Syrup Vehicle), LIPMAX™ (Lecithin & Isopropyl Palmitate), METHYLCELLULOSE, and LIQUIGEL COMPLEX™ (all available from Medisca).

In some variations, the oral suspension vehicle is silica gel, syrup, cellulose, hypromellose or methylcellulose.

Other oral suspension vehicles may include PCCA SUSPENDIT, PCCA MUCOLOX, PCCA SUSPENDIT ANHYDROUS, PCCA-PLUS ORAL SUSPENDING VEHICLE, PCCA POLOXAMER, PCCA SWEET-SF SUGAR FREE SYRUP VEHICLE, PCCA SYRUP VEHICLE, PCCA PRACAMAC, VITAMIN D3(2400 U/ML) IN ALMOND OIL, PCCA SWEETNESS ENHANCER, VITAMIN A PALMITATE (18,000) IN ALMOND OIL AVAILBLE IN 10,000 ALSO, VITAMIN E ACETATE (125U/ML) IN ALMOND OIL, PCAA FIXED OIL SUSPENSION, PCCA LETCITHIN ISOPROPYL SOLUTION, SORBITOL LOLLIPOP**, and PCCA ACACIA SYRUP (all available from PCCA).

Other oral suspension vehicles may include SyrSpend SF Alka Dry, SyrSpend SF Alka Dry Cherry, SyrSpend SF Grape, SyrSpend SF PH4 Dry, SyrSpend SF Pwd Dry, Unispend Anhydrous, medium chain triglycerides, Cherry Syrup, Chew-Hesive, Flavor-Sweet-SF, Flavor-Sweet, Grape Syrup, Methylcellulose, Raspberry Syrup, Simple Syrup (all available from FAGRON).

In some variations, the oral suspension vehicle is a medium chain triglyceride or methylcellulose.

Other oral suspension vehicles may include Oil Corn NF, Gelatin, Polyethylene Glycol, Polyethylene Glycol, 300, NF, Polyethylene Glycol, 1450, NF, Grapeseed oil, Syrup Vehicle, Suspension Vehicle, Syrup Vehicle, Almond Oil, Glycerin, Simpgel 30 (thickening agent), Sepino P 600 (thickening agent), Wetting Agents/Solvents, Butyl Alcohol, NF, Propylene Glycol, USP, DMSO, Mineral Oil Light, Heavy, Glycerin, Isopropyl Alcohol, USP 99% (all available from LETCO).

In some variations, the oral suspension vehicle is Gelatin, almond oil or glycerin.

Other oral suspension vehicles may include SuspendRx Anhydrous (sweetened) Oral Suspension Vehicle w/Bitter Bloc Technology, SuspendRx Anhydrous (unsweetened) Oral Suspension Vehicle w/Bitter Bloc Technology, FOS-ATM Fixed Oil Suspension Vehicle (Unsweetened), Almond Oil, NF (Sweet) (Natural), Medium Chain Triglycerides (MCT) Oil, USP/NF, FOS-ATM (Sweetened) Fixed Oil Suspension Vehicle, SuspendRx™ SF Alka Unflavored-Alkaline Suspension Vehicle for Reconstitution, OraPenn™ SD Anhydrous Sweetened, Suspend S267™ Natural Suspension Base (Powder), ORA-BLEND® (Flavored/Sweetened Oral Suspending Vehicle), OraPenn™ SD Anhydrous UNSWEETENED (*No Sweetener*), ORA-PLUS® (Oral Suspending Vehicle), ORA-BLEND® SF (Flavored/Sweetened/Sugar-Free Oral Suspending Vehicle), ORA-SWEET® (Oral Syrup Vehicle), ORA-SWEET® SF (Sugar-Free Oral Syrup Vehicle), Cottonseed Oil, NF, Grapeseed Oil UltraPure Grade (all available from Specialized Rx).

In some variations, the oral suspension vehicle is Almond Oil, NF (Sweet) (Natural) or Medium Chain Triglycerides (MCT) Oil, USP/NF.

In some variations, the formulation can include a sweetener. Non-limiting examples of sweeteners include advantame, acesulfame, sucralose, aspartane, saccharin, xylitol, glycerin, stevia, thaumatin, honey, erythritol, neotame, agave nectar, maple syrup, molasses, cyclamate, maltitol, sugar alcohols, coconut sugar, sorbitol, sucrose and mannitol.

In some variations, the sweetener is advantame, aspartame, saccharin, stevia, neotame or sucrose.

In some variations, the formulation can include an anti-bitter agent. An anti-bitter agent may be included to alter at least one of the five recognized tastes: sweet, sour, umami, bitter and salty. Anti-bitter agents may be cyclodextrins, polymers, surfactants, microemulsions and microencapsulations.

Non-limiting examples of anti-bitter agents include sodium acetate, sodium gluconate, adenosine-5-monophosphate, magnesium sulfate, zinc sulfate, cyclodextrin, monoeridictyol sodium salt, carboxymethylcellulose sodium salt, catechin, caffeine, propanediol, trilobatin, hesperein dihydrochalcone, gastducin, linguagen, thaumatin, aspartame, monellin and neotame.

In some variations, the anti-bitter agent is magnesium sulfate, zinc sulfate, cyclodextrin, carboxymethylcellulose sodium salt, linguagen, aspartame or neotame.

In some variations, the formulation can include a mucoadhesive agent. Non-limiting examples of mucoadhesive agents include carboxymethylcellulose, alginate, chitosan, polycarbophil, sodium carboxymethylcellulose, hydroxypropylmethylcellulose, carbopol, carbomer polymers, pectin, hyaluronate, acacia, lectins, poloxomer, pluronics, glyceryl monooleate and acrylic acid.

In some variations, the mucoadhesive agent is carboxymethylcellulose, chitosan, carbopol, carbomer polymers, pectin or hyaluronate.

In some variations, the formulation can include a flavoring agent. Non-limiting examples of flavoring agents include manzanate, diacetyl, acetylpropionyl, acetoin, isoamyl acetate, benzaldehyde, bitter almond, cinnamaldehyde, ethyl propionate, methyl anthranilate, limonene, ethyl decadienoate, allyl hexanoate, ethyl maltol, 2,4-dithiapentane, ethylvanillin or methyl salicylate.

In some variations, the flavoring agent is manzanate, bitter almond or cinnamaldehyde.

In some variations, the formulation can include a penetration enhancer. Penetration enhancers are agents that enhance drug delivery through the mucosa by 1) increasing the partitioning of drugs; 2) extracting intercellular lipids; 3) interacting with epithelial protein domains; and/or 4) retention of the drug. Non-limiting examples of penetration enhancers include bile salts, surfactants, fatty acids and derivatives, glycerides, chelators, salicylates, and polymers.

Bile salt that act penetration enhancers may include sodium glycocholate, sodium deoxycholate, sodium taurocholate, sodium fusidate, sodium glycodeoxycholate, or sodium taurodihydrofusidate.

Surfactants that act penetration enhancers may include sodium lauryl sulfate, Brij1-35, lysophosphatidylcholine, dioctyl sodium sulfosuccinate, laurenth-9, polysorbate-80, polyethyleneglycol-8-laurate, or glyceryl monolaurate.

Fatty acids and derivatives that act penetration enhancers may include sorbitan laurate, sodium caprate, sucrose palmitate, lauroyl choline, sodium myristate, or palmitoyl carnitine.

Glycerides that act penetration enhancers may include phospholipids, monohexanoin, medium chain glycerides.

Chelators that act penetration enhancers may include ethylene diamine tetraacetate (EDTA), disodium EDTA.

Salicylates that act penetration enhancers may include salicylic acid, sodium methoxysalicylate, aspirin.

Polymers that act penetration enhancers may include chitosan, polycarbophil, sodium carboxymethylcellulose and their derivatives.

Other penetration enhancers may include Azonel, cyclodextrins, benzalkonium chloride, phenothiazines, nitric acid donors, menthol, Zonula occluden toxin, poly-1-arginines, soybean derivative glucosides, citicholine, a-acid derivative, propylene glycol, salcaprozate sodium (SNAC) or sodium caprate.

In some variations, the penetration enhancer is salcaprozate sodium (SNAC).

In some variations, any of the inactive ingredients described herein may be included in any formulation described herein in amounts of 0 mg up to 10 mg. In some variations, any of the inactive ingredients described herein may be included in any formulation described herein in amounts of up to 20 mg. In some variations, any of the inactive ingredients described herein may be included in any formulation described herein in amounts of up to 30 mg. In some variations, any of the inactive ingredients described herein may be included in any formulation described herein in amounts of up to 40 mg. In some variations, any of the inactive ingredients described herein may be included in any formulation described herein in amounts of up to 50 mg. In some variations, any of the inactive ingredients described herein may be included in any formulation described herein in amounts of up to 60 mg. In some variations, any of the inactive ingredients described herein may be included in any formulation described herein in amounts of up to 70 mg. In some variations, any of the inactive ingredients described herein may be included in any formulation described herein in amounts of up to 80 mg. In some variations, any of the inactive ingredients described herein may be included in any formulation described herein in amounts of up to 90 mg. In some variations, any of the inactive ingredients described herein may be included in any formulation described herein in amounts of up to 100 mg.

In some variations, any of the inactive ingredients described herein may be included in any formulation described herein in amounts of at least 10 mg. In some variations, any of the inactive ingredients described herein may be included in any formulation described herein in amounts of at least 20 mg. In some variations, any of the inactive ingredients described herein may be included in any formulation described herein in amounts of at least 30 mg. In some variations, any of the inactive ingredients described herein may be included in any formulation described herein in amounts of at least 40 mg. In some variations, any of the inactive ingredients described herein may be included in any formulation described herein in amounts of at least 50 mg. In some variations, any of the inactive ingredients described herein may be included in any formulation described herein in amounts of at least 60 mg. In some variations, any of the inactive ingredients described herein may be included in any formulation described herein in amounts of at least 70 mg. In some variations, any of the inactive ingredients described herein may be included in any formulation described herein in amounts of at least 80 mg. In some variations, any of the inactive ingredients described herein may be included in any formulation described herein in amounts of at least 90 mg. In some variations, any of the inactive ingredients described herein may be included in any formulation described herein in amounts of at least 100 mg.

In some variations, a penetration enhancer is included in the sublingual formulations described herein. In some variations, the sublingual formulations include about 5 mg to about 100 mg of penetration enhancer. In some variations, the sublingual formulations include about 10 mg to about 90 mg of penetration enhancer. In some variations, the sublingual formulations include about 20 mg to about 80 mg of penetration enhancer. In some variations, the sublingual formulations include about 20 mg to about 70 mg of penetration enhancer. In some variations, the sublingual formulations include about 20 mg to about 60 mg of penetration enhancer. In some variations, the sublingual formulations include about 20 mg to about 50 mg of penetration enhancer. In some variations, the sublingual formulations include about 20 mg to about 40 mg of penetration enhancer. In some variations, the sublingual formulations include about 20 mg to about 30 mg of penetration enhancer.

In some variations, the sublingual formulations include 0 mg to about 5 mg of penetration enhancer. In some variations, the sublingual formulations include about 5 mg to about 10 mg of penetration enhancer. In some variations, the sublingual formulations include about 10 mg to about 15 mg of penetration enhancer. In some variations, the sublingual formulations include about 15 mg to about 20 mg of penetration enhancer. In some variations, the sublingual formulations include about 20 mg to about 25 mg of penetration enhancer. In some variations, the sublingual formulations include about 25 mg to about 30 mg of penetration enhancer; or about 30 mg to about 35 mg of penetration enhancer. In some variations, the sublingual formulations include about 35 mg to about 40 mg of penetration enhancer. In some variations, the sublingual formulations include about 40 mg to about 45 mg of penetration enhancer. In some variations, the sublingual formulations include about 45 mg to about 50 mg of penetration enhancer. In some variations, the sublingual formulations include about 50 mg to about 55 mg of penetration enhancer. In some variations, the sublingual formulations include about 55 mg to about 60 mg of penetration enhancer. In some variations, the sublingual formulations include about 60 mg to about 65 mg of penetration enhancer. In some variations, the sublingual formulations include about 65 mg to about 70 mg of penetration enhancer. In some variations, the sublingual formulations include about 70 mg to about 75 mg of penetration enhancer. In some variations, the sublingual formulations include about 75 mg to about 80 mg of penetration enhancer. In some variations, the sublingual formulations include about 80 mg to about 85 mg of penetration enhancer. In some variations, the sublingual formulations include about 85 mg to about 90 mg of penetration enhancer. In some variations, the sublingual formulations include about 90 mg to about 95 mg of penetration enhancer. In some variations, the sublingual formulations include about 95 mg to about 100 mg of penetration enhancer.

In some variations, the sublingual formulations include at least about 5 mg of penetration enhancer. In some variations, the sublingual formulations include at least about 10 mg of penetration enhancer. In some variations, the sublingual formulations include at least about 15 mg of penetration enhancer. In some variations, the sublingual formulations include at least about 20 mg of penetration enhancer. In some variations, the sublingual formulations include at least about 25 mg of penetration enhancer. In some variations, the sublingual formulations include at least about 30 mg of penetration enhancer. In some variations, the sublingual formulations include at least about 35 mg of penetration enhancer. In some variations, the sublingual formulations include at least about 40 mg of penetration enhancer. In some variations, the sublingual formulations include at least about 45 mg of penetration enhancer. In some variations, the sublingual formulations include at least about 50 mg of penetration enhancer. In some variations, the sublingual formulations include at least about 55 mg of penetration enhancer. In some variations, the sublingual formulations include at least about 60 mg of penetration enhancer. In some variations, the sublingual formulations include at least about 65 mg of penetration enhancer. In some variations, the sublingual formulations include at least about 70 mg of penetration enhancer. In some variations, the sublingual formulations include at least about 75 mg of penetration enhancer. In some variations, the sublingual formulations include at least about 80 mg of penetration enhancer. In some variations, the sublingual formulations include at least about 85 mg of penetration enhancer. In some variations, the sublingual formulations include at least about 90 mg of penetration enhancer. In some variations, the sublingual formulations include at least about 95 mg of penetration enhancer. In some variations, the sublingual formulations include at least about 100 mg of penetration enhancer.

In some variations, the sublingual formulations include less than or equal to about 5 mg of penetration enhancer. In some variations, the sublingual formulations include less than or equal to about 10 mg of penetration enhancer. In some variations, the sublingual formulations include less than or equal to about 15 mg of penetration enhancer. In some variations, the sublingual formulations include less than or equal to about 20 mg of penetration enhancer. In some variations, the sublingual formulations include less than or equal to about 25 mg of penetration enhancer. In some variations, the sublingual formulations include less than or equal to about 30 mg of penetration enhancer. In some variations, the sublingual formulations include less than or equal to about 35 mg of penetration enhancer. In some variations, the sublingual formulations include less than or equal to about 40 mg of penetration enhancer. In some variations, the sublingual formulations include less than or equal to about 45 mg of penetration enhancer. In some variations, the sublingual formulations include less than or equal to about 50 mg of penetration enhancer. In some variations, the sublingual formulations include less than or equal to about 55 mg of penetration enhancer. In some variations, the sublingual formulations include less than or equal to about 60 mg of penetration enhancer. In some variations, the sublingual formulations include less than or equal to about 65 mg of penetration enhancer. In some variations, the sublingual formulations include less than or equal to about 70 mg of penetration enhancer. In some variations, the sublingual formulations include less than or equal to about 75 mg of penetration enhancer. In some variations, the sublingual formulations include less than or equal to about 80 mg of penetration enhancer. In some variations, the sublingual formulations include less than or equal to about 85 mg of penetration enhancer. In some variations, the sublingual formulations include less than or equal to about 90 mg of penetration enhancer. In some variations, the sublingual formulations include less than or equal to about 95 mg of penetration enhancer. In some variations, the sublingual formulations include less than or equal to about 100 mg of penetration enhancer.

It would be understood that any lower quantity of penetration enhancer can be combined with any upper quantity of penetration enhancer in any combination described herein.

In some variations, the penetration enhancer is SNAC and SNAC is included in the sublingual formulations described herein. In some variations, the sublingual formulations include about 5 mg to about 100 mg of SNAC. In some variations, the sublingual formulations include about 10 mg to about 90 mg of SNAC. In some variations, the sublingual formulations include about 20 mg to about 80 mg of SNAC. In some variations, the sublingual formulations include about 20 mg to about 70 mg of SNAC. In some variations, the sublingual formulations include about 20 mg to about 60 mg of SNAC. In some variations, the sublingual formulations include about 20 mg to about 50 mg of SNAC. In some variations, the sublingual formulations include about 20 mg to about 40 mg of SNAC. In some variations, the sublingual formulations include about 20 mg to about 30 mg of SNAC.

In some variations, the sublingual formulations include 0 mg to about 5 mg of SNAC. In some variations, the sublingual formulations include about 5 mg to about 10 mg of SNAC. In some variations, the sublingual formulations include about 10 mg to about 15 mg of SNAC. In some variations, the sublingual formulations include about 15 mg to about 20 mg of SNAC. In some variations, the sublingual formulations include about 20 mg to about 25 mg of SNAC. In some variations, the sublingual formulations include about 25 mg to about 30 mg of SNAC. In some variations, the sublingual formulations include about 30 mg to about 35 mg of SNAC. In some variations, the sublingual formulations include about 35 mg to about 40 mg of SNAC. In some variations, the sublingual formulations include about 40 mg to about 45 mg of SNAC. In some variations, the sublingual formulations include about 45 mg to about 50 mg of SNAC. In some variations, the sublingual formulations include about 50 mg to about 55 mg of SNAC. In some variations, the sublingual formulations include about 55 mg to about 60 mg of SNAC. In some variations, the sublingual formulations include about 60 mg to about 65 mg of SNAC. In some variations, the sublingual formulations include about 65 mg to about 70 mg of SNAC. In some variations, the sublingual formulations include about 70 mg to about 75 mg of SNAC. In some variations, the sublingual formulations include about 75 mg to about 80 mg of SNAC. In some variations, the sublingual formulations include about 80 mg to about 85 mg of SNAC. In some variations, the sublingual formulations include about 85 mg to about 90 mg of SNAC. In some variations, the sublingual formulations include about 90 mg to about 95 mg of SNAC. In some variations, the sublingual formulations include about 95 mg to about 100 mg of SNAC.

In some variations, the sublingual formulations include at least about 5 mg of SNAC. In some variations, the sublingual formulations include at least about 10 mg of SNAC. In some variations, the sublingual formulations include at least about 15 mg of SNAC. In some variations, the sublingual formulations include at least about 20 mg of SNAC. In some variations, the sublingual formulations include at least about 25 mg of SNAC. In some variations, the sublingual formulations include at least about 30 mg of SNAC. In some variations, the sublingual formulations include at least about 35 mg of SNAC. In some variations, the sublingual formulations include at least about 40 mg of SNAC. In some variations, the sublingual formulations include at least about 45 mg of SNAC. In some variations, the sublingual formulations include at least about 50 mg of SNAC. In some variations, the sublingual formulations include at least about 55 mg of SNAC. In some variations, the sublingual formulations include at least about 60 mg of SNAC. In some variations, the sublingual formulations include at least about 65 mg of SNAC. In some variations, the sublingual formulations include at least about 70 mg of SNAC. In some variations, the sublingual formulations include at least about 75 mg of SNAC. In some variations, the sublingual formulations include at least about 80 mg of SNAC. In some variations, the sublingual formulations include at least about 85 mg of SNAC. In some variations, the sublingual formulations include at least about 90 mg of SNAC. In some variations, the sublingual formulations include at least about 95 mg of SNAC. In some variations, the sublingual formulations include at least about 100 mg of SNAC.

In some variations, the sublingual formulations include less than or equal to about 5 mg of SNAC. In some variations, the sublingual formulations include less than or equal to about 10 mg of SNAC. In some variations, the sublingual formulations include less than or equal to about 15 mg of SNAC. In some variations, the sublingual formulations include less than or equal to about 20 mg of SNAC. In some variations, the sublingual formulations include less than or equal to about 25 mg of SNAC. In some variations, the sublingual formulations include less than or equal to about 30 mg of SNAC. In some variations, the sublingual formulations include less than or equal to about 35 mg of SNAC. In some variations, the sublingual formulations include less than or equal to about 40 mg of SNAC. In some variations, the sublingual formulations include less than or equal to about 45 mg of SNAC. In some variations, the sublingual formulations include less than or equal to about 50 mg of SNAC. In some variations, the sublingual formulations include less than or equal to about 55 mg of SNAC. In some variations, the sublingual formulations include less than or equal to about 60 mg of SNAC. In some variations, the sublingual formulations include less than or equal to about 65 mg of SNAC. In some variations, the sublingual formulations include less than or equal to about 70 mg of SNAC. In some variations, the sublingual formulations include less than or equal to about 75 mg of SNAC. In some variations, the sublingual formulations include less than or equal to about 80 mg of SNAC. In some variations, the sublingual formulations include less than or equal to about 85 mg of SNAC. In some variations, the sublingual formulations include less than or equal to about 90 mg of SNAC. In some variations, the sublingual formulations include less than or equal to about 95 mg of SNAC. In some variations, the sublingual formulations include less than or equal to about 100 mg of SNAC.

It would be understood that any lower quantity of SNAC can be combined with any upper quantity of SNAC in any combination described herein.

### C. Methods of Use

The formulations described herein are used as a medicament. In some variations, the formulations described herein are used in the treatment or prevention of diabetes and/or obesity.

It will be appreciated that the active ingredient(s) for use as a sublingual, buccal or rectal pharmaceutical, may be described as a method of administration or alternatively be described as use of a composition in the manufacture of such a pharmaceutical.

It will be appreciated that the method of administration described herein may alternatively be described as a composition for use as a sublingual, buccal or rectal pharmaceutical, alternatively use of a composition in the manufacture of a sublingual, buccal or rectal pharmaceutical. The method of administration described herein may alternatively be described as semaglutide for use as a sublingual, buccal or rectal pharmaceutical, alternatively use of semaglutide in the manufacture of a sublingual, buccal or rectal pharmaceutical.

Analogously, the use of semaglutide described herein may alternatively be described as a method of administration or use of semaglutide in the manufacture of a sublingual, buccal or rectal pharmaceutical. In some variations, the terms “dosing regimen” and “method of administration” are used interchangeably herein. Herein, in some variations, the term “use” includes a composition for use, e.g., “use in medicine” includes a “composition for use in medicine”. In some variations, the term “method” as used herein includes a method of administration, e.g., a method of oral administration.

The method of administration of the invention comprises sublingual, buccal or rectal therapy. In some variations, the method comprises treatment or prevention of diabetes and/or obesity. In some variations, the method comprises a method of managing weight. In some variations, the method comprises control of glucose. In some variations, the method comprises a method of reduction of cardiovascular events in adults with diabetes.

In some variations, the method or use comprises (e.g., the semaglutide formulations of the invention may be used for the following medical treatments):
(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;
(iii) prevention and/or treatment of eating disorders, such as obesity, e.g., by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying;
(iv) managing weight; controlling and/or reducing glucose levels; controlling and/or reducing A1C levels; and reducing cardiovascular events in adults with diabetes; and
(v) prevention and/or treatment of insulin resistance; prevention and/or treatment of metabolic syndrome; or prevention and/or treatment of poly-cyctic ovarian syndrome (PCOS).

In some variations, the indication is (i). In some variations, the indication is (ii). In some variations, the indication is (iii). In some variations, the indication is (iv). In some variations, the indication is (v). In some variations, the indication is type 2 diabetes and/or obesity.

In some variations, the method or use comprises prevention, treatment, reduction and/or induction in one or more diseases or conditions defined herein. In some variations, the indication is (i) and (ii). In some variations, the indication is (i) and (iii). In some variations, the indication is (i) and (iv). In some variations, the indication is (i) and (v). In some variations, the indication is (ii) and (iii). In some variations, the indication is (ii) and (iv). In some variations, the indication is (ii) and (v). In some variations, the indication is (iii) and (iv). In some variations, the indication is (iii) and (v). In some variations, the indication is (iv) and (v). In some variations, the indication is (i), (ii), (iii), (iv) and (v).

In some variations, the invention comprises administration of an effective amount of a semaglutide formulation described herein. In some variations, the invention relates to administration of an effective amount of a sublingual semaglutide formulation described herein. In some variations, the invention relates to administration of an effective amount of a buccal semaglutide formulation described herein. In some variations, the invention relates to administration of an effective amount of a rectal semaglutide formulation described herein.

In some variations, as used herein, specific values given in relation to numbers or intervals may be understood as the specific value or as about the specific value.

In some variations, the sublingual formulations described herein, when administered to a human patient, lead to steady state concentrations of semaglutide that is comparable to those of FDA approved semaglutide products, while using less semaglutide. In some variations, the steady state concentration of semaglutide after administration of a sublingual formulation described herein is between about 25 nmol/L to about 250 nmol/L. In some variations, the steady state concentration of semaglutide after administration of a sublingual formulation described herein is between about 30 nmol/L to about 225 nmol/L. In some variations, the steady state concentration of semaglutide after administration of a sublingual formulation described herein is between about 35 nmol/L to about 200 nmol/L. In some variations, the steady state concentration of semaglutide after administration of a sublingual formulation described herein is between about 40 nmol/L to about 150 nmol/L. In some variations, the steady state concentration of semaglutide after administration of a sublingual formulation described herein is between about 50 nmol/L to about 100 nmol/L.

In some variations, the steady state concentration of semaglutide after administration of a sublingual formulation described herein is at least about 25 nmol/L. In some variations, the steady state concentration of semaglutide after administration of a sublingual formulation described herein is at least about 30 nmol/L. In some variations, the steady state concentration of semaglutide after administration of a sublingual formulation described herein is at least about 35 nmol/L. In some variations, the steady state concentration of semaglutide after administration of a sublingual formulation described herein is at least about 40 nmol/L. In some variations, the steady state concentration of semaglutide after administration of a sublingual formulation described herein is at least about 45 nmol/L. In some variations, the steady state concentration of semaglutide after administration of a sublingual formulation described herein is at least about 50 nmol/L. In some variations, the steady state concentration of semaglutide after administration of a sublingual formulation described herein is at least about 55 nmol/L. In some variations, the steady state concentration of semaglutide after administration of a sublingual formulation described herein is at least about 60 nmol/L. In some variations, the steady state concentration of semaglutide after administration of a sublingual formulation described herein is at least about 65 nmol/L. In some variations, the steady state concentration of semaglutide after administration of a sublingual formulation described herein is at least about 70 nmol/L. In some variations, the steady state concentration of semaglutide after administration of a sublingual formulation described herein is at least about 75 nmol/L. In some variations, the steady state concentration of semaglutide after administration of a sublingual formulation described herein is at least about 80 nmol/L. In some variations, the steady state concentration of semaglutide after administration of a sublingual formulation described herein is at least about 85 nmol/L. In some variations, the steady state concentration of semaglutide after administration of a sublingual formulation described herein is at least about 90 nmol/L. In some variations, the steady state concentration of semaglutide after administration of a sublingual formulation described herein is at least about 95 nmol/L. In some variations, the steady state concentration of semaglutide after administration of a sublingual formulation described herein is at least about 100 nmol/L. In some variations, the steady state concentration of semaglutide after administration of a sublingual formulation described herein is at least about 105 nmol/L. In some variations, the steady state concentration of semaglutide after administration of a sublingual formulation described herein is at least about 110 nmol/L. In some variations, the steady state concentration of semaglutide after administration of a sublingual formulation described herein is at least about 115 nmol/L. In some variations, the steady state concentration of semaglutide after administration of a sublingual formulation described herein is at least about 120 nmol/L. In some variations, the steady state concentration of semaglutide after administration of a sublingual formulation described herein is at least about 125 nmol/L. In some variations, the steady state concentration of semaglutide after administration of a sublingual formulation described herein is at least about 130 nmol/L. In some variations, the steady state concentration of semaglutide after administration of a sublingual formulation described herein is at least about 135 nmol/L. In some variations, the steady state concentration of semaglutide after administration of a sublingual formulation described herein is at least about 140 nmol/L. In some variations, the steady state concentration of semaglutide after administration of a sublingual formulation described herein is at least about 145 nmol/L. In some variations, the steady state concentration of semaglutide after administration of a sublingual formulation described herein is at least about 150 nmol/L. In some variations, the steady state concentration of semaglutide after administration of a sublingual formulation described herein is at least about 155 nmol/L. In some variations, the steady state concentration of semaglutide after administration of a sublingual formulation described herein is at least about 160 nmol/L. In some variations, the steady state concentration of semaglutide after administration of a sublingual formulation described herein is at least about 165 nmol/L. In some variations, the steady state concentration of semaglutide after administration of a sublingual formulation described herein is at least about 170 nmol/L. In some variations, the steady state concentration of semaglutide after administration of a sublingual formulation described herein is at least about 175 nmol/L. In some variations, the steady state concentration of semaglutide after administration of a sublingual formulation described herein is at least about 180 nmol/L. In some variations, the steady state concentration of semaglutide after administration of a sublingual formulation described herein is at least about 185 nmol/L. In some variations, the steady state concentration of semaglutide after administration of a sublingual formulation described herein is at least about 190 nmol/L. In some variations, the steady state concentration of semaglutide after administration of a sublingual formulation described herein is at least about 195 nmol/L. In some variations, the steady state concentration of semaglutide after administration of a sublingual formulation described herein is at least about 200 nmol/L. In some variations, the steady state concentration of semaglutide after administration of a sublingual formulation described herein is at least about 205 nmol/L. In some variations, the steady state concentration of semaglutide after administration of a sublingual formulation described herein is at least about 210 nmol/L. In some variations, the steady state concentration of semaglutide after administration of a sublingual formulation described herein is at least about 215 nmol/L. In some variations, the steady state concentration of semaglutide after administration of a sublingual formulation described herein is at least about 220 nmol/L. In some variations, the steady state concentration of semaglutide after administration of a sublingual formulation described herein is at least about 225 nmol/L. In some variations, the steady state concentration of semaglutide after administration of a sublingual formulation described herein is at least about 230 nmol/L. In some variations, the steady state concentration of semaglutide after administration of a sublingual formulation described herein is at least about 235 nmol/L. In some variations, the steady state concentration of semaglutide after administration of a sublingual formulation described herein is at least about 240 nmol/L. In some variations, the steady state concentration of semaglutide after administration of a sublingual formulation described herein is at least about 245 nmol/L. In some variations, the steady state concentration of semaglutide after administration of a sublingual formulation described herein is at least about 250 nmol/L. In some variations, the steady state concentration of semaglutide after administration of a sublingual formulation described herein is at least about 255 nmol/L. In some variations, the steady state concentration of semaglutide after administration of a sublingual formulation described herein is at least about 260 nmol/L. In some variations, the steady state concentration of semaglutide after administration of a sublingual formulation described herein is at least about 265 nmol/L. In some variations, the steady state concentration of semaglutide after administration of a sublingual formulation described herein is at least about 270 nmol/L. In some variations, the steady state concentration of semaglutide after administration of a sublingual formulation described herein is at least about 275 nmol/L.

In some variations, the steady state concentration of semaglutide after administration of a sublingual formulation described herein is less than or equal to 25 nmol/L. In some variations, the steady state concentration of semaglutide after administration of a sublingual formulation described herein is less than or equal to about 30 nmol/L. In some variations, the steady state concentration of semaglutide after administration of a sublingual formulation described herein is less than or equal to about 35 nmol/L. In some variations, the steady state concentration of semaglutide after administration of a sublingual formulation described herein is less than or equal to about 40 nmol/L. In some variations, the steady state concentration of semaglutide after administration of a sublingual formulation described herein is less than or equal to about 45 nmol/L. In some variations, the steady state concentration of semaglutide after administration of a sublingual formulation described herein is less than or equal to about 50 nmol/L. In some variations, the steady state concentration of semaglutide after administration of a sublingual formulation described herein is less than or equal to about 55 nmol/L. In some variations, the steady state concentration of semaglutide after administration of a sublingual formulation described herein is less than or equal to about 60 nmol/L. In some variations, the steady state concentration of semaglutide after administration of a sublingual formulation described herein is less than or equal to about 65 nmol/L. In some variations, the steady state concentration of semaglutide after administration of a sublingual formulation described herein is less than or equal to about 70 nmol/L. In some variations, the steady state concentration of semaglutide after administration of a sublingual formulation described herein is less than or equal to about 75 nmol/L. In some variations, the steady state concentration of semaglutide after administration of a sublingual formulation described herein is less than or equal to about 80 nmol/L. In some variations, the steady state concentration of semaglutide after administration of a sublingual formulation described herein is less than or equal to about 85 nmol/L. In some variations, the steady state concentration of semaglutide after administration of a sublingual formulation described herein is less than or equal to about 90 nmol/L. In some variations, the steady state concentration of semaglutide after administration of a sublingual formulation described herein is less than or equal to about 95 nmol/L. In some variations, the steady state concentration of semaglutide after administration of a sublingual formulation described herein is less than or equal to about 100 nmol/L. In some variations, the steady state concentration of semaglutide after administration of a sublingual formulation described herein is less than or equal to about 105 nmol/L. In some variations, the steady state concentration of semaglutide after administration of a sublingual formulation described herein is less than or equal to about 110 nmol/L. In some variations, the steady state concentration of semaglutide after administration of a sublingual formulation described herein is less than or equal to about 115 nmol/L. In some variations, the steady state concentration of semaglutide after administration of a sublingual formulation described herein is less than or equal to about 120 nmol/L. In some variations, the steady state concentration of semaglutide after administration of a sublingual formulation described herein is less than or equal to about 125 nmol/L. In some variations, the steady state concentration of semaglutide after administration of a sublingual formulation described herein is less than or equal to about 130 nmol/L. In some variations, the steady state concentration of semaglutide after administration of a sublingual formulation described herein is less than or equal to about 135 nmol/L. In some variations, the steady state concentration of semaglutide after administration of a sublingual formulation described herein is less than or equal to about 140 nmol/L. In some variations, the steady state concentration of semaglutide after administration of a sublingual formulation described herein is less than or equal to about 145 nmol/L. In some variations, the steady state concentration of semaglutide after administration of a sublingual formulation described herein is less than or equal to about 150 nmol/L. In some variations, the steady state concentration of semaglutide after administration of a sublingual formulation described herein is less than or equal to about 155 nmol/L. In some variations, the steady state concentration of semaglutide after administration of a sublingual formulation described herein is less than or equal to about 160 nmol/L. In some variations, the steady state concentration of semaglutide after administration of a sublingual formulation described herein is less than or equal to about 165 nmol/L. In some variations, the steady state concentration of semaglutide after administration of a sublingual formulation described herein is less than or equal to about 170 nmol/L. In some variations, the steady state concentration of semaglutide after administration of a sublingual formulation described herein is less than or equal to about 175 nmol/L. In some variations, the steady state concentration of semaglutide after administration of a sublingual formulation described herein is less than or equal to about 180 nmol/L. In some variations, the steady state concentration of semaglutide after administration of a sublingual formulation described herein is less than or equal to about 185 nmol/L. In some variations, the steady state concentration of semaglutide after administration of a sublingual formulation described herein is less than or equal to about 190 nmol/L. In some variations, the steady state concentration of semaglutide after administration of a sublingual formulation described herein is less than or equal to about 195 nmol/L. In some variations, the steady state concentration of semaglutide after administration of a sublingual formulation described herein is less than or equal to about 200 nmol/L. In some variations, the steady state concentration of semaglutide after administration of a sublingual formulation described herein is less than or equal to about 205 nmol/L. In some variations, the steady state concentration of semaglutide after administration of a sublingual formulation described herein is less than or equal to about 210 nmol/L. In some variations, the steady state concentration of semaglutide after administration of a sublingual formulation described herein is less than or equal to about 215 nmol/L. In some variations, the steady state concentration of semaglutide after administration of a sublingual formulation described herein is less than or equal to about 220 nmol/L. In some variations, the steady state concentration of semaglutide after administration of a sublingual formulation described herein is less than or equal to about 225 nmol/L. In some variations, the steady state concentration of semaglutide after administration of a sublingual formulation described herein is less than or equal to about 230 nmol/L. In some variations, the steady state concentration of semaglutide after administration of a sublingual formulation described herein is less than or equal to about 235 nmol/L. In some variations, the steady state concentration of semaglutide after administration of a sublingual formulation described herein is less than or equal to about 240 nmol/L. In some variations, the steady state concentration of semaglutide after administration of a sublingual formulation described herein is less than or equal to about 245 nmol/L. In some variations, the steady state concentration of semaglutide after administration of a sublingual formulation described herein is less than or equal to about 250 nmol/L. In some variations, the steady state concentration of semaglutide after administration of a sublingual formulation described herein is less than or equal to about 255 nmol/L. In some variations, the steady state concentration of semaglutide after administration of a sublingual formulation described herein is less than or equal to about 260 nmol/L. In some variations, the steady state concentration of semaglutide after administration of a sublingual formulation described herein is less than or equal to about 265 nmol/L. In some variations, the steady state concentration of semaglutide after administration of a sublingual formulation described herein is less than or equal to about 270 nmol/L. In some variations, the steady state concentration of semaglutide after administration of a sublingual formulation described herein is less than or equal to about 275 nmol/L.

In some variations, the steady state concentration of semaglutide after administration of a sublingual formulation described herein is between about 25 nmol/L to about 30 nmol/L. In some variations, the steady state concentration of semaglutide after administration of a sublingual formulation described herein is between about 30 nmol/L to about 35 nmol/L. In some variations, the steady state concentration of semaglutide after administration of a sublingual formulation described herein is between about 35 nmol/L to about 40 nmol/L. In some variations, the steady state concentration of semaglutide after administration of a sublingual formulation described herein is between about 40 nmol/L to about 45 nmol/L. In some variations, the steady state concentration of semaglutide after administration of a sublingual formulation described herein is between about 45 nmol/L to about 50 nmol/L. In some variations, the steady state concentration of semaglutide after administration of a sublingual formulation described herein is between about 50 nmol/L to about 55 nmol/L. In some variations, the steady state concentration of semaglutide after administration of a sublingual formulation described herein is between about 55 nmol/L to about 60 nmol/L. In some variations, the steady state concentration of semaglutide after administration of a sublingual formulation described herein is between about 60 nmol/L to about 65 nmol/L. In some variations, the steady state concentration of semaglutide after administration of a sublingual formulation described herein is between about 65 nmol/L to about 70 nmol/L. In some variations, the steady state concentration of semaglutide after administration of a sublingual formulation described herein is between about 70 nmol/L to about 75 nmol/L. In some variations, the steady state concentration of semaglutide after administration of a sublingual formulation described herein is between about 75 nmol/L to about 80 nmol/L. In some variations, the steady state concentration of semaglutide after administration of a sublingual formulation described herein is between about 80 nmol/L to about 85 nmol/L. In some variations, the steady state concentration of semaglutide after administration of a sublingual formulation described herein is between about 85 nmol/L to about 90 nmol/L. In some variations, the steady state concentration of semaglutide after administration of a sublingual formulation described herein is between about 90 nmol/L to about 95 nmol/L. In some variations, the steady state concentration of semaglutide after administration of a sublingual formulation described herein is between about 95 nmol/L to about 100 nmol/L. In some variations, the steady state concentration of semaglutide after administration of a sublingual formulation described herein is between about 100 nmol/L to about 105 nmol/L. In some variations, the steady state concentration of semaglutide after administration of a sublingual formulation described herein is between about 105 nmol/L to about 110 nmol/L. In some variations, the steady state concentration of semaglutide after administration of a sublingual formulation described herein is between about 110 nmol/L to about 115 nmol/L. In some variations, the steady state concentration of semaglutide after administration of a sublingual formulation described herein is between about 115 nmol/L to about 120 nmol/L. In some variations, the steady state concentration of semaglutide after administration of a sublingual formulation described herein is between about 120 nmol/L to about 125 nmol/L. In some variations, the steady state concentration of semaglutide after administration of a sublingual formulation described herein is between about 125 nmol/L to about 130 nmol/L. In some variations, the steady state concentration of semaglutide after administration of a sublingual formulation described herein is between about 130 nmol/L to about 135 nmol/L. In some variations, the steady state concentration of semaglutide after administration of a sublingual formulation described herein is between about 135 nmol/L to about 140 nmol/L. In some variations, the steady state concentration of semaglutide after administration of a sublingual formulation described herein is between about 140 nmol/L to about 145 nmol/L. In some variations, the steady state concentration of semaglutide after administration of a sublingual formulation described herein is between about 145 nmol/L to about 150 nmol/L. In some variations, the steady state concentration of semaglutide after administration of a sublingual formulation described herein is between about 150 nmol/L to about 155 nmol/L. In some variations, the steady state concentration of semaglutide after administration of a sublingual formulation described herein is between about 155 nmol/L to about 160 nmol/L. In some variations, the steady state concentration of semaglutide after administration of a sublingual formulation described herein is between about 160 nmol/L to about 165 nmol/L. In some variations, the steady state concentration of semaglutide after administration of a sublingual formulation described herein is between about 165 nmol/L to about 170 nmol/L. In some variations, the steady state concentration of semaglutide after administration of a sublingual formulation described herein is between about 170 nmol/L to about 175 nmol/L. In some variations, the steady state concentration of semaglutide after administration of a sublingual formulation described herein is between about 175 nmol/L to about 180 nmol/L. In some variations, the steady state concentration of semaglutide after administration of a sublingual formulation described herein is between about 180 nmol/L to about 185 nmol/L. In some variations, the steady state concentration of semaglutide after administration of a sublingual formulation described herein is between about 185 nmol/L to about 190 nmol/L. In some variations, the steady state concentration of semaglutide after administration of a sublingual formulation described herein is between about 190 nmol/L to about 195 nmol/L. In some variations, the steady state concentration of semaglutide after administration of a sublingual formulation described herein is between about 195 nmol/L to about 200 nmol/L. In some variations, the steady state concentration of semaglutide after administration of a sublingual formulation described herein is between about 200 nmol/L to about 205 nmol/L. In some variations, the steady state concentration of semaglutide after administration of a sublingual formulation described herein is between about 205 nmol/L to about 210 nmol/L. In some variations, the steady state concentration of semaglutide after administration of a sublingual formulation described herein is between about 210 nmol/L to about 215 nmol/L. In some variations, the steady state concentration of semaglutide after administration of a sublingual formulation described herein is between about 215 nmol/L to about 220 nmol/L. In some variations, the steady state concentration of semaglutide after administration of a sublingual formulation described herein is between about 220 nmol/L to about 225 nmol/L. In some variations, the steady state concentration of semaglutide after administration of a sublingual formulation described herein is between about 225 nmol/L to about 230 nmol/L. In some variations, the steady state concentration of semaglutide after administration of a sublingual formulation described herein is between about 230 nmol/L to about 235 nmol/L. In some variations, the steady state concentration of semaglutide after administration of a sublingual formulation described herein is between about 235 nmol/L to about 240 nmol/L. In some variations, the steady state concentration of semaglutide after administration of a sublingual formulation described herein is between about 240 nmol/L to about 245 nmol/L. In some variations, the steady state concentration of semaglutide after administration of a sublingual formulation described herein is between about 245 nmol/L to about 250 nmol/L. In some variations, the steady state concentration of semaglutide after administration of a sublingual formulation described herein is between about 250 nmol/L to about 255 nmol/L. In some variations, the steady state concentration of semaglutide after administration of a sublingual formulation described herein is between about 255 nmol/L to about 260 nmol/L. In some variations, the steady state concentration of semaglutide after administration of a sublingual formulation described herein is between about 260 nmol/L to about 265 nmol/L. In some variations, the steady state concentration of semaglutide after administration of a sublingual formulation described herein is between about 265 nmol/L to about 270 nmol/L. In some variations, the steady state concentration of semaglutide after administration of a sublingual formulation described herein is between about 270 nmol/L to about 275 nmol/L.

It would be understood that any lower quantity of steady state concentration of semaglutide can be combined with any upper quantity of steady state concentration of semaglutide in any combination described herein.

### EMBODIMENTS OF THE DISCLOSURE

A first embodiment of the invention relates to a sublingual, buccal or rectal formulation comprising 0.25 mg-5.0 mg semaglutide and one or more inactive components.

A second embodiment of the invention relates to the sublingual, buccal or rectal formulation according to the first embodiments, wherein the formulation comprises naltrexone.

A third embodiment of the invention relates to the sublingual, buccal or rectal formulation according to the first embodiments, wherein the formulation comprises vitamin B₁₂ or methylated vitamin B₁₂.

A fourth embodiment of the invention relates to the sublingual, buccal or rectal formulation according to the first embodiments, wherein the formulation comprises naltrexone and vitamin B₁₂ or methylated vitamin B₁₂.

A fifth embodiment of the invention relates to the sublingual, buccal or rectal formulation according to any of the previous embodiments, wherein the formulation comprises at least one compound that reduces cravings.

A sixth embodiment of the invention relates to the sublingual, buccal or rectal formulation according to the fifth embodiments, wherein the at least one compound that reduces cravings is naltrexone, bupoprion, diethylpropion, 5-htp, b vitamin, magnesium picolinate, chromium picolinate, green tea, linoleic acid, caffeine, an amphetamine or a stimulant.

A seventh embodiment of the invention relates to the sublingual, buccal or rectal formulation according to the sixth embodiments, wherein the at least one compound that reduces cravings is phentermine, phendimetrazine, naltrexone, diethylpropion, or bupropion.

An eighth embodiment of the invention relates to the sublingual, buccal or rectal formulation according to any of the previous embodiments, wherein the one or more inactive components comprise an anhydrous base that is a medium chain triglyceride, acacia, coconut oil, cottonseed oil, mineral oil, polyethylene glycol, glycerin, olive oil, vegetable oil or almond oil.

A ninth embodiment of the invention relates to the sublingual, buccal or rectal formulation according to the eighth embodiments, wherein the anhydrous base is a medium chain triglyceride, vegetable oil or glycerin.

A tenth embodiment of the invention relates to the sublingual, buccal or rectal formulation according to any of the previous embodiments, wherein the one or more inactive components comprise an oral suspension vehicle that is cherry syrup, grape syrup, methylcellulose, a medium chain triglyceride, raspberry syrup, polyethylene glycol or grapeseed oil.

An eleventh embodiment of the invention relates to the sublingual, buccal or rectal formulation according to the tenth embodiments, wherein the oral suspension vehicle is a medium chain triglyceride or methylcellulose.

A twelfth embodiment of the invention relates to the sublingual, buccal or rectal formulation according to any of the previous embodiments, wherein the one or more inactive components comprise a sweetener that is advantame, acesulfame, sucralose, aspartane, saccharin, xylitol, glycerin, stevia, thaumatin, honey, erythritol, neotame, agave nectar, maple syrup, molasses, cyclamate, maltitol, sugar alcohols, coconut sugar, sorbitol, sucrose or mannitol.

A thirteenth embodiment of the invention relates to the sublingual, buccal or rectal formulation according to the twelfth embodiments, wherein the sweetener is advantame, aspartame, saccharin, stevia, neotame or sucrose.

A fourteenth embodiment of the invention relates to the sublingual, buccal or rectal formulation according to any of the previous embodiments, wherein the one or more inactive components comprise an anti-bitter agent that is sodium acetate, sodium gluconate, adenosine-5-monophosphate, magnesium sulfate, zinc sulfate, cyclodextrin, monoeridictyol sodium salt, carboxymethylcellulose sodium salt, catechin, caffeine, propanediol, trilobatin, hesperein dihydrochalcone, gastducin, linguagen, thaumatin, aspartame, monellin and neotame.

A fifteenth embodiment of the invention relates to the sublingual, buccal or rectal formulation according to the fourteenth embodiments, wherein the anti-bitter agent is magnesium sulfate, zinc sulfate, cyclodextrin, carboxymethylcellulose sodium salt, linguagen, aspartame or neotame.

A sixteenth embodiment of the invention relates to the sublingual, buccal or rectal formulation according to any of the previous embodiments, wherein the one or more inactive components comprise a mucoadhesive agent that is carboxymethyl-cellulose, alginate, chitosan, polycarbophil, sodium carboxymethylcellulose, hydroxypropyl-methylcellulose, carbopol, carbomer polymers, pectin, hyaluronate, acacia, lectins, poloxomer, pluronics, glyceryl monooleate and acrylic acid.

A seventeenth embodiment of the invention relates to the sublingual, buccal or rectal formulation according to the sixteenth embodiments, wherein the mucoadhesive agent is carboxymethylcellulose, chitosan, carbopol, carbomer polymers, pectin or hyaluronate.

An eighteenth embodiment of the invention relates to the sublingual, buccal or rectal formulation according to any of the previous embodiments, wherein the one or more inactive components comprise a flavoring agent that is manzanate, diacetyl, acetylpropionyl, acetoin, isoamyl acetate, benzaldehyde, bitter almond, cinnamaldehyde, ethyl propionate, methyl anthranilate, limonene, ethyl decadienoate, allyl hexanoate, ethyl maltol, 2,4-dithiapentane, ethylvanillin or methyl salicylate.

A nineteenth embodiment of the invention relates to the sublingual, buccal or rectal formulation according to the eighteenth embodiments, wherein the flavoring agent is manzanate, bitter almond or cinnamaldehyde.

A twentieth embodiment of the invention relates to the sublingual, buccal or rectal formulation according to any of the previous embodiments, wherein the one or more inactive components comprise a penetration enhancer that is sodium glycocholate, sodium deoxycholate, sodium taurocholate, sodium fusidate, sodium glycodeoxycholate, sodium taurodihydrofusidate, sodium lauryl sulfate, Brij 1-35, lysophosphatidylcholine, dioctyl sodium sulfosuccinate, laurenth-9, polysorbate-80, polyethyleneglycol-8-laurate, glyceryl monolaurate, sorbitan laurate, sodium caprate, sucrose palmitate, lauroyl choline, sodium myristate, palmitoyl carnitine, phospholipids, monohexanoin, medium chain glycerides, ethylene diamine tetraacetate (EDTA), disodium EDTA, salicylic acid, sodium methoxysalicylate, aspirin, chitosan, polycarbophil, sodium carboxymethylcellulose and their derivatives, Azonel, cyclodextrins, benzalkonium chloride, phenothiazines, nitric acid donors, menthol, Zonula occluden toxin, poly-l-arginines, soybean derivative glucosides, citicholine, a-acid derivative, propylene glycol, salcaprozate sodium (SNAC) or sodium caprate.

A twenty-first embodiment of the invention relates to the sublingual, buccal or rectal formulation according to the twentieth embodiments, wherein the penetration enhancer is SNAC.

A twenty-second embodiment of the invention relates to the sublingual, buccal or rectal formulation according to any of the previous embodiments, wherein the formulation comprises 0.5 mg-1.5 mg semaglutide.

A twenty-third embodiment of the invention relates to the sublingual, buccal or rectal formulation according to any one of the first-twenty-first embodiments, wherein the formulation comprises 1.5 mg-2.5 mg semaglutide.

A twenty-fourth embodiment of the invention relates to the sublingual, buccal or rectal formulation according to any one of the first-twenty-first embodiments, wherein the formulation comprises 2.5 mg-3.5 mg semaglutide.

A twenty-fifth embodiment of the invention relates to the sublingual, buccal or rectal formulation according to any one of the first-twenty-first embodiments, wherein the formulation comprises 3.5 mg-4.5 mg semaglutide.

A twenty-sixth embodiment of the invention relates to the sublingual, buccal or rectal formulation according to any one of the first-twenty-first embodiments, wherein the formulation comprises up to 5.0 mg semaglutide.

A twenty-seventh embodiment of the invention relates to the sublingual, buccal or rectal formulation according to any one of the preceding embodiments, wherein the formulation comprises 0.0005 mg-50.0 mg naltrexone.

A twenty-eighth embodiment of the invention relates to the sublingual, buccal or rectal formulation according to any one of the first-twenty-sixth embodiments, wherein the formulation comprises 1.5 mg-4.0 mg naltrexone.

A twenty-ninth embodiment of the invention relates to the sublingual, buccal or rectal formulation according to any one of the first-twenty-sixth embodiments, wherein the formulation comprises 8.0 mg-32.0 mg naltrexone.

A thirtieth embodiment of the invention relates to the sublingual, buccal or rectal formulation according to any one of the first-twenty-sixth embodiments, wherein the formulation comprises about 50.0 mg naltrexone.

A thirty-first embodiment of the invention relates to the sublingual, buccal or rectal formulation according to any one of the preceding embodiments, wherein the formulation comprises 100.0 mcg-2500.0 mcg Vitamin B₁₂ or methylated Vitamin B₁₂.

A thirty-second embodiment of the invention relates to the sublingual, buccal or rectal formulation according to any one of the first-thirtieth embodiments, wherein the formulation comprises 500.0 mcg-2000.0 mcg Vitamin B₁₂ or methylated Vitamin B₁₂.

A thirty-third embodiment of the invention relates to the sublingual, buccal or rectal formulation according to any one of the first-thirtieth embodiments, wherein the formulation comprises 1000.0 mcg-1500.0 mcg Vitamin B₁₂ or methylated Vitamin B₁₂.

A thirty-fourth embodiment of the invention relates to the sublingual, buccal or rectal formulation according to the first embodiments, wherein the formulation is a sublingual tablet or suspension.

A thirty-fifth embodiment of the invention relates to the sublingual, buccal or rectal formulation according to the first embodiments, wherein the formulation is a sublingual tablet.

A thirty-sixth embodiment of the invention relates to the sublingual, buccal or rectal formulation according to the first embodiments, wherein the formulation is a sublingual suspension.

A thirty-seventh embodiment of the invention relates to the sublingual, buccal or rectal formulation according to the first embodiments, wherein the formulation is a buccal tablet, buccal wafer, buccal troche or buccal lozenge.

A thirty-eighth embodiment of the invention relates to the sublingual, buccal or rectal formulation according to the first embodiments, wherein the formulation is a buccal tablet.

A thirty-ninth embodiment of the invention relates to the sublingual, buccal or rectal formulation according to the first embodiments, wherein the formulation is a buccal wafer.

A fortieth embodiment of the invention relates to the sublingual, buccal or rectal formulation according to the first embodiments, wherein the formulation is a buccal troche.

A forty-first embodiment of the invention relates to the sublingual, buccal or rectal formulation according to the first embodiments, wherein the formulation is a buccal lozenge.

A forty-second embodiment of the invention relates to the sublingual, buccal or rectal formulation according to the first embodiments, wherein the formulation is a suppository.

A forty-third embodiment of the invention relates to a method for treating diabetes and/or obesity in a subject in need of such treatment, said method comprising administering to said subject a therapeutically effective amount of the sublingual, buccal or rectal formulation of any one of the preceding embodiments.

A forty-fourth embodiment of the invention relates to a method for treating diabetes and/or obesity in a subject in need of such treatment according to the forty-third embodiments, wherein said method comprises administering the formulation sublingually.

A forty-fifth embodiment of the invention relates to a method for treating diabetes and/or obesity in a subject in need of such treatment according to the forty-third embodiments, wherein said method comprises administering the formulation buccally.

A forty-sixth embodiment of the invention relates to a method for treating diabetes and/or obesity in a subject in need of such treatment according to the forty-third embodiments, wherein said method comprises administering the formulation rectally.

A forty-seventh embodiment of the invention relates to a method for managing weight in a subject in need of such treatment, said method comprising administering to said subject a therapeutically effective amount of the sublingual, buccal or rectal formulation of any one of the first through forty-second embodiments.

A forty-eighth embodiment of the invention relates to a method for managing weight in a subject in need of such treatment according to the forty-seventh embodiments, wherein said method comprises administering the formulation sublingually.

A forty-ninth embodiment of the invention relates to a method for managing weight in a subject in need of such treatment according to the forty-seventh embodiments, wherein said method comprises administering the formulation buccally.

A fiftieth embodiment of the invention relates to a method for treating diabetes and/or obesity in a subject in need of such treatment according to the forty-seventh embodiments, wherein said method comprises administering the formulation rectally.

A fifty-first embodiment of the invention relates to a method for controlling glucose in a subject in need of such treatment, said method comprising administering to said subject a therapeutically effective amount of the sublingual, buccal or rectal formulation of any one of the first through forty-second embodiments.

A fifty-second embodiment of the invention relates to a method for controlling glucose in a subject in need of such treatment according to the fifty-first embodiments, wherein said method comprises administering the formulation sublingually.

A fifty-third embodiment of the invention relates to a method for controlling glucose in a subject in need of such treatment according to the fifty-first embodiments, wherein said method comprises administering the formulation buccally.

A fifty-fourth embodiment of the invention relates to a method for controlling glucose in a subject in need of such treatment according to the fifty-first embodiments, wherein said method comprises administering the formulation rectally.

A fifty-fifth embodiment of the invention relates to a method for reducing cardiovascular events in an adult with diabetes in a subject in need of such treatment, said method comprising administering to said subject a therapeutically effective amount of the sublingual, buccal or rectal formulation of any one of the first through forty-second embodiments.

A fifty-sixth embodiment of the invention relates to a method for reducing cardiovascular events in an adult with diabetes in a subject in need of such treatment according to the fifty-fifth embodiments, wherein said method comprises administering the formulation sublingually.

A fifty-seventh embodiment of the invention relates to a method for reducing cardiovascular events in an adult with diabetes in a subject in need of such treatment according to the fifty-fifth embodiments, wherein said method comprises administering the formulation buccally.

A fifty-eighth embodiment of the invention relates to a method for reducing cardiovascular events in an adult with diabetes in a subject in need of such treatment according to the fifty-fifth embodiments, wherein said method comprises administering the formulation rectally.

A fifty-ninth embodiment of the invention relates to the method of any one of the forty-third through fifty-eighth embodiments, wherein the sublingual, buccal or rectal formulation is administered at least once a day.

A sixtieth embodiment of the invention relates to the method of any one of the forty-third through fifty-ninth embodiments, wherein the sublingual, buccal or rectal formulation is administered at least twice a day.

A sixty-first embodiment of the invention relates to the method of any one of the forty-third through fifty-eighth embodiments, wherein the sublingual, buccal or rectal formulation is administered at least once a week.

A sixty-second embodiment of the invention relates to the method of any one of the forty-third through fifty-eighth embodiments, wherein the sublingual, buccal or rectal formulation is administered at least twice a week.

The following Examples are provided to illustrate some of the concepts described within this disclosure. While the Examples are considered to provide an embodiments, it should not be considered to limit the more general embodiments described herein.

### EXAMPLES

### Example 1

Sublingual formulations comprising semaglutide or semaglutide and at least one of naltrexone and vitamin B₁₂. Such sublingual formulations are described below in Table 1.

TABLE 1
Sublingual Formulations Comprising Semaglutide

| No. | Ingredients | Amounts | Formulation |
|---|---|---|---|
| 1 | SEMAGLUTIDE | 1 mg/0.35 ml | Suspension |
| 2 | SEMAGLUTIDE | 2 mg/0.35 ml | Suspension |
| 3 | SEMAGLUTIDE | 1.5 mg/0.35 ml | Suspension |
| 4 | SEMAGLUTIDE | 2 mg | TROCHE |
| 5 | SEMAGLUTIDE/Vitamin B₁₂ | 1 mg/500 mcg/0.35 ml | Suspension |
| 6 | SEMAGLUTIDE/Vitamin B₁₂ | 2 mg/500 mcg/0.35 ml | Suspension |
| 7 | SEMAGLUTIDE/Vitamin B₁₂ | 1.5 mg/500 mcg/0.35 ml | Suspension |
| 8 | SEMAGLUTIDE/Vitamin B₁₂ | 0.25 mg/500 mcg/0.35 ml | Suspension |
| 9 | SEMAGLUTIDE/Vitamin B₁₂ | 0.5 mg/500 mcg/0.35 ml | Suspension |
| 10 | SEMAGLUTIDE/Vitamin B₁₂ | 1 mg/500 mcg | TROCHE |
| | (MINT/MARSHMALLOW) | | |
| 11 | SEMAGLUTIDE/Vitamin B₁₂ | 2 mg/500 mcg | TROCHE |
| | (MINT/MARSHMALLOW) | | |
| 12 | SEMAGLUTIDE/Vitamin B₁₂ | 3 mg/500 mcg | TROCHE |
| | (MINT/MARSHMALLOW) | | |
| 13 | SEMAGLUTIDE/Vitamin B₁₂ | 1.5 mg/500 mcg | TROCHE |
| | (MINT/MARSHMALLOW) | | |
| 14 | SEMAGLUTIDE/Vitamin B₁₂ | 2 mg/500 mcg | RD Tablet |
| | RDT (MINT/RASPBERRY) | | |
| 15 | SEMAGLUTIDE/Vitamin B₁₂ | 1 mg/500 mcg | RD Tablet |
| | RDT (MINT/RASPBERRY) | | |
| 16 | SEMAGLUTIDE/NALTREXONE | 2 mg/8 mg | TROCHE |
| | (MINT/MARSHMALLOW) | | |
| 17 | SEMAGLUTIDE/NALTREXONE/ | 2 mg/8 mg/500 mcg/0.35 ml | Suspension |
| | Vitamin B₁₂ | | |
| 18 | SEMAGLUTIDE/NALTREXONE/ | 2 mg/4 mg/500 mcg/0.35 ml | Suspension |
| | Vitamin B₁₂ | | |
| 19 | SEMAGLUTIDE/NALTREXONE/ | 1.5 mg/4 mg/500 mcg/0.35 ml | Suspension |
| | Vitamin B₁₂ | | |
| 20 | SEMAGLUTIDE/NALTREXONE/ | 1 mg/4 mg/500 mcg/0.35 ml | Suspension |
| | Vitamin B₁₂ | | |
| 21 | SEMAGLUTIDE/NALTREXONE/ | 2 mg/16 mg/500 mcg/0.35 ml | Suspension |
| | Vitamin B₁₂ | | |
| 22 | SEMAGLUTIDE/NALTREXONE/ | 3 mg/4 mg/500 mcg/0.7 ml | Suspension |
| | Vitamin B₁₂ | | |
| 23 | SEMAGLUTIDE/NALTREXONE/ | 3 mg/16 mg/500 mcg | TROCHE |
| | Vitamin B₁₂ (MINT/MARSHMALLOW) | | |
| 24 | SEMAGLUTIDE/NALTREXONE/ | 2 mg/4 mg/500 mcg | TROCHE |
| | Vitamin B₁₂ (MINT/MARSHMALLOW) | | |
| 25 | SEMAGLUTIDE/NALTREXONE/ | 1 mg/4 mg/500 mcg | TROCHE |
| | Vitamin B₁₂ (MINT/MARSHMALLOW) | | |
| 26 | SEMAGLUTIDE/NALTREXONE/ | 3 mg/4 mg/500 mcg | TROCHE |
| | Vitamin B₁₂ (MINT/MARSHMALLOW) | | |
| 27 | SEMAGLUTIDE/NALTREXONE/ | 2 mg/16 mg/500 mcg | TROCHE |
| | Vitamin B₁₂ (MINT/MARSHMALLOW) | | |
| 28 | SEMAGLUTIDE/NALTREXONE/ | 1 mg/16 mg/500 mcg | TROCHE |
| | Vitamin B₁₂ (MINT/MARSHMALLOW) | | |
| 29 | SEMAGLUTIDE/NALTREXONE/ | 2 mg/4 mg/500 mcg | RD Tablet |
| | Vitamin B₁₂ RDT (MINT/RASPBERRY) | | |
| 30 | SEMAGLUTIDE/NALTREXONE/ | 1 mg/4 mg/500 mcg | RD Tablet |
| | Vitamin B₁₂ RDT (MINT/RASPBERRY) | | |

### General Preparation Methods-Troches

Troches described in Table 1 were made by the following methods.
1. Active powder and silica gel were weighed on balance and covered with a boat until ready to use.
2. An appropriate size beaker and spin bar were tared on a balance.
3. The troche base was weighed into tared beaker.
4. The beaker was placed on a hot plate and the temperature to 200° C. As the troche base melts, a glass stir rod was used to mix the base until stir bar could be used. The stir bar was used once base sufficiently melted.
5. Once the troche base is fully melted and spinning as a uniform color, the beaker and hot plate were moved into the hood with the powders from step 1.
6. While under the hood, these powders from step 1 were added and then spun at a setting of 6 with no heat until the powder was fully dispersed.
7. The mixture was homogenized until uniform and remixed as needed.
8. The TEMPMIX troche molds were laid out on a counter along with a plastic scrapper.
9. Once the powders dispersed, the warm mixture was spread over and filled into each cavity.
10. The heat gun was used to reheat troches in the mold as they cool to keep troches level. If air bubbles appeared, the troche tops were reheated with heat gun.
11. The troches were allowed to cool at room temperature and weight checks were performed.

### General Preparation Methods—Suspensions

Suspensions described in Table 1 were made by the following methods.
1. Tablets of 14 mg Rybelsus were crushed into a fine powder using a mortor and pestle and sieved if necessary.
2. The bitterness reducing agent, stevia, cyanocobalamin and if needed, naltrexone, were weighed.
3. The powders were added to the mortor and pestle and mixed.
4. A small amount of olive oil was added to make a smooth paste.
5. The liquids (peppermint oil and grape oil) were measured and added to the paste.
6. The paste was transferred to a beaker.
7. The final volume with obtained by addition of olive oil.
8. The suspension was homogenized by spinning at 20-25 rpms for 60 seconds before placing the suspension in its final container.

### General Preparation Methods—RD Tablets

Readily Dissolving Tablets (RDT) described in Table 1 were made by the following methods.
1. The active ingredient powder was weighed.
2. Tablets of 14 mg Rybelsus were counted and crushed and, if needed, and sieved through a 40-50 mesh
3. The RDT powder, flavor and bitter stop, if needed, were weighed. A sweetener was included in the RDT powder.
4. In a Resodyne mixer (RAM), the powders were blended by EMP or mortar and pestle and mixed with geometric dilution.
5. A 96 mold cavity was filled by tapping and pressing the powder into each cavity. The tapping and pressing was repeated as needed to ensure each cavity was completely filled. A tamper may be used if necessary.
6. The powder blend was heated to 105-110° C. in convection oven for 10-15 minutes. Care was taken to ensure the blend was not overheated.
7. The RDTs were allowed to cool and removed from the mold.

### Example 2. Steady State Bioavailability—Suspension

Steady state concentration levels of semaglutide were determined for sublingual suspensions described in Example 1 above.

Patients in this cohort were administered a sublingual suspension of semaglutide/naltrexone/cyanocobalamin 2 mg/4 mg/500 mcg (e.g., Formulation #18 in Example 1) for 4 days. The patients placed 0.35 ml of suspension under the tongue every morning and held the medication for 20-30 minutes under the tongue. The patient swallowed what was left after that time and waited 30 minutes to eat or drink anything. Some patients became moody using Formulation #18 and changed to a sublingual suspension of semaglutide/cyanocobalamin 1 mg/500 mcg (e.g., Formulation #5 in Example 1) daily. Blood samples of each patient were then taken at steady state after 35 days of administration. No doses were missed. Some patients were GLP-1 naïve.

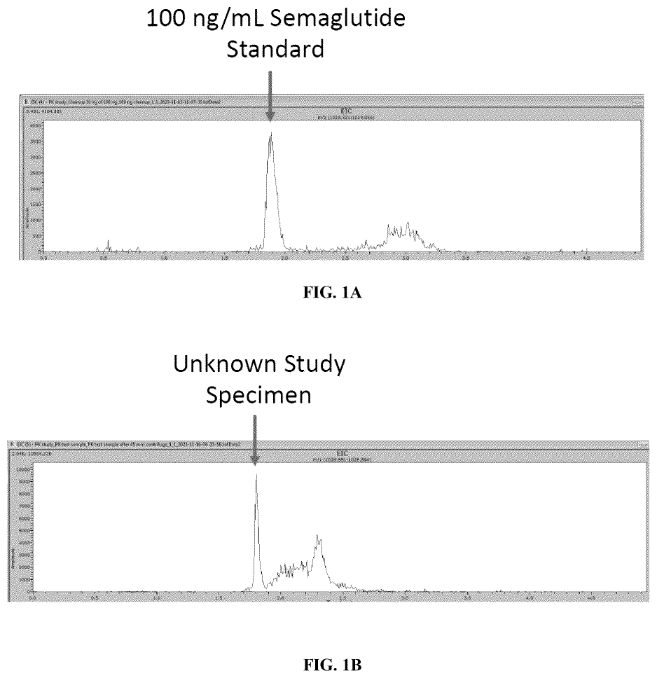

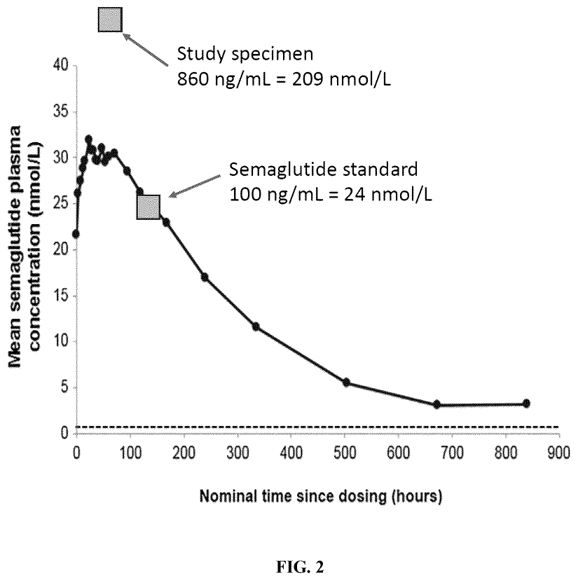

Steady state plasma concentration of semaglutide is shown in FIGS. 1 and 2. FIG. 1A shows the ultra-high performance liquid chromatography-quadrupole time-of-flight mass spectra (UHPLC/TOF-MS) of the 100 ng/mL semaglutide standard. FIG. 1B shows the UHPLC/TOF-MS of the semaglutide study specimen. The steady state plasma concentration of the semaglutide study specimen was determined to be 860 ng/ml. Such a steady state concentration is equivalent to 209 nmol/L.

FIG. 2 shows a comparison of the kinetic profile of the bioavailability of semaglutide over time. As can be seen, the steady state specimen has a higher steady state concentration than the standard and. The steady state specimen also has a higher steady state concentration than the published levels of either Rybelsus tablets or Ozempic injection.

### Example 3. Steady State Bioavailability—Troche

Steady state concentration levels of semaglutide were determined for sublingual troches described in Example 1 above.

Patients in this cohort were administered a sublingual troche (e.g., Formulation #10 in Example 1) that was placed under the tongue and allowed to dissolve once a day in the morning for 4 consecutive days. The time to dissolve averaged 13 minutes. Patients didn't eat or drink for 30 minutes after the troche was dissolved. Some patients experienced mild nausea. Some patients experienced moodiness and fatigue on day 3 of medication administration.

Similar blood concentration occurred by the second and third blood draw. There was observed a slower ramp up to similar concentrations by day 2 and 3 with the troche administration. The slower ramp up of semaglutide concentration via troche administration is beneficial since the slow ramp up is believed to limit adverse events and the need for titration.

Also, it is noted that each tablet of Rybelsus includes 300 mg of SNAC. Therefore, for each mg of semaglutide in the troche, about 21.43 mg of SNAC is included. The reduced amount of SNAC would indicate less potential side effects with semaglutide administration via troche.

Patients in this cohort were determined to have an average steady state concentration of semaglutide between about 45 nmol/L to about 50 nmol/L.

### Example 4. Steady State Bioavailability—Troche

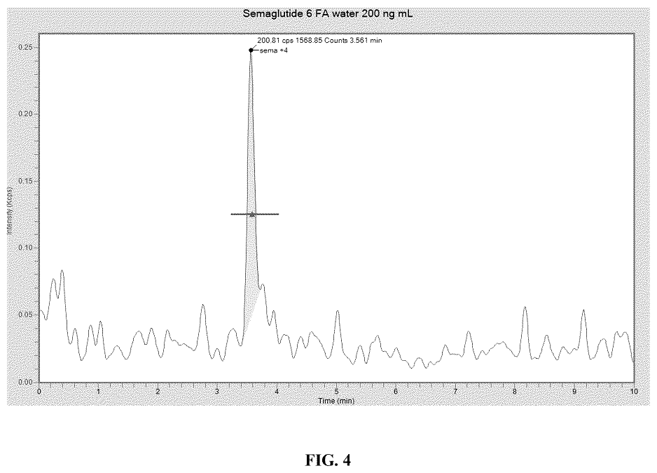

Steady state concentration levels of semaglutide were determined for sublingual troches described in Example 1 above. Results are shown in FIG. 4.

Patients in this cohort were administered a sublingual troche comprising semaglutide/cyanocobalamin 1 mg/500 mcg troche (e.g., Formulation #10 in Example 1) daily until steady state (more than 35 days). The troches were dissolved under the tongue each morning. Once the troche dissolved, the patients didn't eat or drink for 30 minutes after.

### Example 5. Steady State Bioavailability—Comparison of Troche of Example 1 vs. Intravenously Administered Semaglutide

Patients were given a troche (e.g., Formulation #10 in Example 1). Steady state bioavailability was compared to injectable semaglutide.

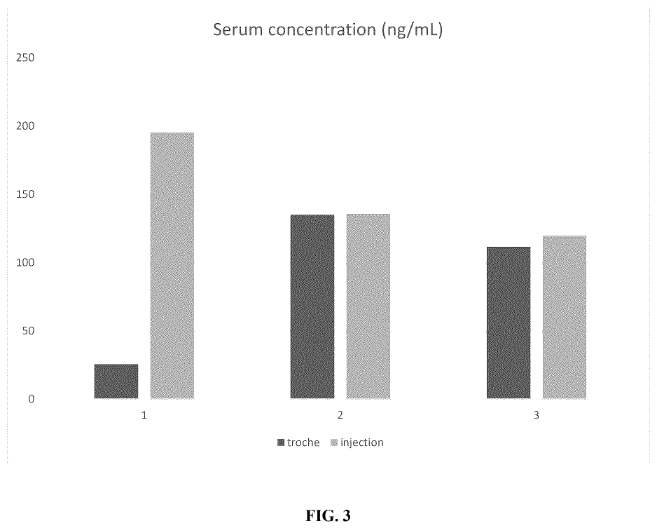

As shown in FIG. 3, the steady state bioavailability of the injectable formulation is higher than that of the troche after the first drawing of blood. However, the bioavailability of the injectable formulation tails off after the second and third drawings of blood, while the that of the troche increases such that both the injectable formulation and the troche formulation have similar concentration after the second and third drawings of blood. The first drawing of blood for each cohort was on the day of initial administration of the formulation. The time between the first and second drawings of blood was two days for each cohort. The time between the second and third drawings of blood was also two days for each cohort.

### Example 6. Dosing Simulations

Dosing simulations were conducted to predict the level of bioavailability of semaglutide formulations. Steady state concentration of semaglutide from FDA approved semaglutide injectable formulations were simulated based on weekly administration. Steady state concentration of semaglutide from the troche formulations described herein were simulated based on bi-daily administration. Based on simulation data (not shown), troche formulations described herein demonstrate a controllable dosing regime that is better able to manage semaglutide concentrations.

A comparison to FDA approved semaglutide injectable formulations is informative. The dosing simulation data show that if a patient being administered an FDA approved semaglutide injectable formulation has an adverse event, that adverse event is likely to be present for about a week. With the troche formulations described herein, once similar concentrations of semaglutide are achieved that cause the adverse event, the dosing and side effect profile can be adjusted.

### CONCLUSIONS

Daily dosing of sublingual formulations comprising semaglutide leads to higher plasma concentration in the steady state. These results are surprising because large molecular weight molecules (such as semaglutide) are difficult to make bioavailable via oral administration (such as sublingual administration). For instance, steady state concentration of semaglutide in a sublingual suspension (in a single patient) are at least about 4 times higher, e.g., 209 nmol/L, than those of FDA approved semaglutide injectable formulations, e.g., 23-51 nmol/L. See efaidnbmnnnibpcajpcglclefindmkaj/https://www.accessdata.fda.gov/drugsatfda_docs/nda/2017/209637Orig1s000ClinPharmR.pdf. For instance, steady state concentration of semaglutide in a sublingual suspension are at least about 4 times higher (e.g., 209 nmol/L) than those of FDA approved semaglutide oral tablets (e.g., 30-50 nmol/L). See efaidnbmnnnibpcajpcglclefindmkaj/https://www.accessdata.fda.gov/drugsatfda_docs/nda/2019/213051Origls000ClinPharmR.pdf. The sublingual formulations comprise far less semaglutide amounts than the FDA approved products.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A sublingual, buccal, or rectal formulation comprising:
0.25 mg-3.0 mg semaglutide;
one or more B vitamins; and
one or more inactive components.

2. The formulation of claim 1, comprising naltrexone.

3. The formulation of claim 1, wherein the one or more B vitamins comprise vitamin B₁₂ or methylated vitamin B₁₂.

4. The formulation of claim 1, comprising povidone and salcaprozate sodium (SNAC).

5. The sublingual formulation of claim 1, comprising magnesium stearate and microcrystalline cellulose (MCC).

6-19. (canceled)

20. The sublingual formulation of claim 1 comprising a penetration enhancer selected from the group consisting of sodium glycocholate, sodium deoxycholate, sodium taurocholate, sodium fusidate, sodium glycodeoxycholate, sodium taurodihydrofusidate, sodium lauryl sulfate, Brij1-35, lysophosphatidylcholine, dioctyl sodium sulfosuccinate, laurenth-9, polysorbate-80, polyethyleneglycol-8-laurate, glyceryl monolaurate, sorbitan laurate, sodium caprate, sucrose palmitate, lauroyl choline, sodium myristate, palmitoyl carnitine, phospholipids, monohexanoin, medium chain glycerides, ethylene diamine tetraacetate (EDTA), disodium EDTA, salicylic acid, sodium methoxysalicylate, aspirin, chitosan, polycarbophil, sodium carboxymethylcellulose and their derivatives, Azonel, cyclodextrins, benzalkonium chloride, phenothiazines, nitric acid donors, menthol, Zonula occluden toxin, poly-l-arginines, soybean derivative glucosides, citicholine, a-acid derivative, propylene glycol, salcaprozate sodium (SNAC) or sodium caprate.

21. The sublingual formulation of claim 20, wherein the penetration enhancer is SNAC.

22-26. (canceled)

27. The sublingual formulation of claim 1, comprising 0.0005 mg-50.0 mg naltrexone.

28-30. (canceled)

31. The sublingual formulation of claim 1, comprising 100.0 mcg-2500.0 mcg Vitamin B₁₂ or methylated Vitamin B₁₂.

32-33. (canceled)

34. The sublingual formulation of claim 1, wherein the formulation is a tablet or suspension.

35-36. (canceled)

37. A buccal formulation comprising:
less than or equal to 0.25 mg semaglutide; and
one or more inactive components.

38-108. (canceled)

109. The formulation of claim 1, wherein the formulation is a sublingual formulation comprising:
about 1 mg-3 mg of semaglutide;
about 4 mg-32 mg naltrexone;
about 500 mcg-1000 mcg of Vitamin B₁₂; and
0 mg-10 mg of one or more inactive components.

110. The sublingual formulation of claim 109, wherein the formulation is a suspension or a tablet.

111. (canceled)

112. The formulation of claim 1, wherein the formulation is a buccal formulation comprising
about 1 mg-2 mg of semaglutide;
about 4 mg-16 mg naltrexone;
about 500 mcg-1000 mcg of Vitamin B₁₂; and
0 mg-10 mg of one or more inactive components.

113. The buccal formulation of claim 112, wherein the formulation is a suspension or a tablet.

114. (canceled)

115. The formulation of claim 1, wherein the formulation is a rectal formulation comprising
about 1 mg-2 mg of semaglutide;
about 4 mg-16 mg naltrexone;
about 500 mcg-1000 mcg of Vitamin B₁₂; and
0 mg-10 mg of one or more inactive components.

116. The rectal formulation of claim 115, wherein the formulation is a suspension.

117-133. (canceled)

134. A method for treating diabetes and/or obesity in a subject in need of such treatment, said method comprising:
administering to said subject a therapeutically effective amount of the formulation of claim 1.

135-139. (canceled)

140. A method for controlling glucose in a subject in need of such treatment, said method comprising:
administering to said subject a therapeutically effective amount of the formulation of claim 1.

141-142. (canceled)

143. A method for reducing cardiovascular events in an adult with diabetes in a subject in need of such treatment, said method comprising:
administering to said subject a therapeutically effective amount of the formulation of claim 1.

144-149. (canceled)
